(19) United States
(12) Patent Application Publication
(10) Pub. No.: US 2026/0055376 A1
(43) Pub. Date: Feb. 26, 2026

(54) ENGINEERED HUMAN CARDIAC TISSUE

(71) Applicants: Murdoch Children's Research Institute, Parkville, Victoria (AU); The Council of the Queensland Institute of Medical Research, Herston, Queensland (AU)

(72) Inventors: Enzo Porello, Parkville, Victoria (AU); Christian Brizard, Parkville, Victoria (AU); James Edward Hudson, Herston, Queensland (AU); Ellen Frances Mathieson, Herston, Queensland (AU); Andrew Laskary, Herston, Queensland (AU); Liam Reynolds, Herston, Queensland (AU)

(73) Assignees: Murdoch Children's Research Institute, Parkville, Victoria (AU); The Council of the Queensland Institute of Medical Research, Herston, Queensland (AU)

(21) Appl. No.: 18/996,968

(22) PCT Filed: Jul. 21, 2023
(86) PCT No.: PCT/AU2023/050668

§ 371 Date: Jan. 17, 2025

(30) Foreign Application Priority Data
Jul. 21, 2022 (AU) 2022902048

(51) Int. Cl. C12N 5/071; A61L 27/38; C12N 5/077

(52) U.S. Cl. CPC C12N 5/069; A61L 27/3808; C12N 5/0661; C12N 5/0692; A61L 2430/20; C12N 2500/24; C12N 2500/99; C12N 2501/115; C12N 2501/135; C12N 2501/33; C12N 2501/415; C12N 2501/727; C12N 2506/1315; C12N 2510/00; C12N 2533/56; C12N 2539/00

(57) ABSTRACT

The present application relates to methods for the generation of engineered human pluripotent stem cell-derived cardiac tissue, engineered cardiac tissue produced by such methods and methods for their use.

### FIELD

The present application relates to methods for the generation of engineered human pluripotent stem cell-derived cardiac tissue, engineered cardiac tissue produced by such methods and methods for their use.

### BACKGROUND

Despite many breakthroughs in cardiovascular surgery and medicine, heart attacks and heart failure remain the most prominent health challenges. Heart disease is also the leading cause of death and disability in children, affecting up to 1 in 100 live births. In adults diagnosed with congestive heart failure (CHF), studies have found that mortality is approximately 50% within 5 years of this diagnosis. An emerging and alarming trend is the sharp rise in the number of children and adults with CHD hospitalised due to heart failure. Surgical advances over the past 20 years have dramatically increased survival rates in childhood heart disease patients, with more than 85% of children with CHD now living into adulthood. As a result, CHD is now considered a life-long disease.

Heart failure is characterized by loss or dysfunction of cardiomyocytes, whether because of ischemic heart disease, hypertensive heart disease, a congenital heart disease or idiopathic cardiomyopathy. The adult heart muscle is unable to regenerate and the damaged myocardial tissue is replaced with non-contractile scar tissue. Currently, heart failure can only be resolved by heart transplantation, but the gap between the number of donors and the number of patients with heart failure requiring transplantation is growing. Current treatment options are inadequate and new approaches to radically change patient trajectories are imperative.

The directed differentiation of human pluripotent stem cells (hPSCs), including both human embryonic stem cells (hES) and human induced pluripotent stem cells (hiPS), to distinct cellular endpoints has enabled the generation of organoid models of a variety of human tissues, including cardiac tissue. Tissue engineering techniques have sought to generate stem-cell derived functional cardiac tissue that may be used for disease modelling and treatment. However, existing methods and protocols for the generation of engineered heart tissues fail to accurately recapitulate native heart tissue composition and function (e.g. contractility). Various protocols reported in the literature generate cardiac tissue that is comprised of cardiomyocytes and stromal cells (fibroblasts) (see. e.g. WO2015/040142), but they typically lack endothelial cells, which represent the most abundant non-myocyte cell population in the human heart, and other vascular cells (smooth muscle cells). While populations of cardiomyocytes, stromal cells and endothelial cells can be readily obtained through different separate differentiation protocols and then combined, the combination or mixing of cell types from separate differentiation protocols introduces undesirable complexity and variability.

Furthermore, current protocols and cell culture media formulations attempt to promote advanced maturation and contractility of tissue engineered cardiac tissue by enhancing oxidative metabolism (Mills et al., 2017. Proc. Natl. Acad. Sci. USA, 114(40):E8372-E8381), however oxidative metabolism may not be optimal for survival and engraftment following implantation into a hypoxic environment in vivo. Oxidative metabolism also inhibits cardiomyocyte proliferation (Mills et al., 2017) which may not be optimal for growth of the tissue following implantation in vivo.

There remains a need in the art for a method for generating pluripotent stem cell-derived cardiac tissue that overcomes these disadvantages.

### SUMMARY OF INVENTION

Through detailed studies the inventors have developed a protocol for the generation of engineered human stem-cell derived cardiac tissue comprising appropriate diversity of cell types found in native heart tissue, and may be derived from a single differentiation protocol.

According to a first aspect, the present invention provides a method for producing engineered human cardiac tissue comprising endothelial cells, the method comprising culturing a cell suspension comprising a population of human pluripotent stem cell (hPSC) culture-derived cardiomyocytes and non-cardiomyocyte cells in the presence of a FGFR1 agonist and a PDGFR agonist for a time and under conditions sufficient to produce engineered human cardiac tissue comprising endothelial cells.

According to a second aspect, the present invention provides a method for producing engineered human cardiac tissue, the method comprising the steps of: i) mixing a cell suspension comprising a population of human pluripotent stem cell (hPSC) culture-derived cardiomyocytes and non-cardiomyocyte cells with a flowable hydrogel composition, wherein said cell suspension is in a serum-free medium; ii) loading the flowable hydrogel composition comprising said cell suspension into a mold and incubating the hydrogel for a time and under conditions sufficient to solidify the hydrogel; and iii) culturing the hydrogel from step ii) in serum free medium; wherein the population of human pluripotent stem cell (hPSC) culture-derived cardiomyocytes and non-cardiomyocyte cells is contacted with an FGFR1 agonist and a PDGFR agonist to thereby provide engineered human cardiac tissue, wherein said tissue comprises cardiomyocytes, stromal cells/fibroblasts and endothelial cells.

According to a third aspect, the present invention provides engineered human cardiac tissue comprising at least about 30% hPSC-derived cardiomyocytes, about 35% or less hPSC-derived stromal cells/fibroblasts and about 30% or less hPSC-derived endothelial cells, wherein the aforementioned cells are located throughout a hydrogel composition.

According to a fourth aspect, the present invention provides engineered human cardiac tissue comprising hPSC-derived cardiomyocytes, hPSC-derived stromal cells/fibroblasts and hPSC-derived endothelial cells, wherein the aforementioned cells are derived from a single differentiation process, wherein the aforementioned cells are located throughout a hydrogel composition.

According to a fifth aspect, the present invention provides engineered human cardiac tissue produced according to the method of the first aspect.

According to a sixth aspect, the present invention provides a method for analysing the biological effect of at least one test compound or bioactive agent on cardiac cells, comprising contacting engineered human cardiac tissue of any one of the second, third or fourth aspects with the test compound or bioactive agent, incubating the tissue in the presence of the test compound or bioactive agent, and analysing the biological effect.

According to a seventh aspect, the present invention provides engineered human cardiac tissue of any one of the second, third or fourth aspects for use as an implant in the treatment of diseased or damaged cardiac tissue in a subject in need thereof.

According to an eighth aspect, the present invention provides a method for treating diseased or damaged cardiac tissue in a subject in need thereof comprising implanting the engineered human cardiac tissue of any one of the second, third or fourth aspects in said subject.

According to a ninth aspect, the present invention provides use of engineered human cardiac tissue of any one of the second, third or fourth aspects in the manufacture of a medicament for the treatment of diseased or damaged cardiac tissue in a subject in need thereof.

### BRIEF DESCRIPTION OF DRAWINGS

FIG. 1 shows: A) a schematic overview of cardiac differentiation and tissue engineering protocol. Human pluripotent stem cells are differentiated into cardiac cells and on day 15 the cells are enzymatically dissociated and mixed with a hydrogel before being placed in a custom-designed PDMS mold. The tissues are cultured on the custom-designed scaffold for about 7 days before being removed from the mold for implantation. B) Photo of the Bioengineered Heart Tissue Patch (BHTP). C-E) immunofluorescent and histological images of fixed, paraffin-embedded, and sectioned BHTPs. Immunofluorescent images demonstrates that cells within the fibrin matrix express cardiac specific markers including α-actinin, myosin light chain 2 (MLC2v) (panel C), and cardiac troponin T (cTnT) (panel D). Nuclei in C) and D) are labelled with Hoechst. E) histological image is a hematoxylin and eosin stain illustrating the overall tissue architecture of BHTPs and the cardiac cells distributed within them.

FIG. 2 shows a single cell RNA Sequencing UMAP Plot showing cell clusters present in BHTP (Serum free (sf) “new” media). Cell clusters present in the BHTP include cardiomyocytes (purple), endothelial cells (red), fibroblasts/stromal cells (green), smooth muscle cells (blue) and cardiac progenitor cells (gold).

FIG. 3 shows a heatmap showing expression of cell type-specific marker genes across broad cell types. CM (Prlf), Cardiomyocyte (proliferative); CM, Cardiomyocyte; Endo, Endothelial; Fibro/Stromal, Fibroblast/Stromal cells; and Smc, Smooth muscle cell; Cardiac Progenitors.

FIG. 4 shows UMAP plots showing the expression of Cardiomyocyte (proliferative) markers (ANLN, TOP2A and CDK1) across samples.

FIG. 5 shows UMAP plots showing the expression of Cardiomyocyte markers (TNNT2, ACTN2 and MYH7) across samples.

FIG. 6 shows UMAP plots showing the expression of Endothelial markers (PECAM1, ENG, EMCN) across samples.

FIG. 7 shows UMAP plots showing the expression of Smooth Muscle Cell markers (TAGLN and ACTA2) across samples.

FIG. 8 shows UMAP plots showing the expression of Fibroblast/Stromal markers (LAMB1, COL1A2 and COL1A1) across samples.

FIG. 9 shows BHTP (new media) cardiomyocytes are characterized by reduced expression of genes involved in oxidation reduction processes and fatty acid oxidation. A) Single cell RNA Sequencing UMAP Plot showing cell clusters present in BHTP (new media) compared to BHTP (old media), and a cardiomyocyte cluster used for downstream gene set enrichment analysis (GSEA). Sub-cluster of proliferative cardiomyocytes, CM(Prlf), shown in light blue. B) Heat map showing expression of genes associated with gene ontology (GO) biological processes associated with oxidation reduction, fatty acid oxidation and muscle contraction. Markers for these processes are up-regulated during human development (primary cardiomyocytes) from fetal to adult stages. Markers are also elevated in BHTP old media vs new media. C) Dot plots showing exemplar genes for biological processes demonstrating reduced expression of oxidative stress markers in BHTP new media.

FIG. 10 shows relative gene expression for glycolysis (PKM2) and oxidation-reduction markers (ALDH1A1, AGMO, DPYD, GPX3, CYP1B1, PLIN5) following pseudo-bulk analysis of cardiomyocytes in snRNA-seq data obtained from engineered human cardiac tissues prepared in SF medium or MM (“old”) medium. Data presented as log2 relative gene expression normalized to the housekeeping gene TUBA1A.

FIG. 11 shows new media BHTP retains a higher proportion of proliferative cardiomyocytes. Pie charts showing the percentage of proliferative cardiomyocytes as a proportion of the total cell population. New media rep1: 2.66%, new media rep2: 3.98%, old media rep2: 1.12%. The percentage of proliferative cardiomyocytes as a proportion of the total cardiomyocyte population is; new media rep1: 7.55%, new media rep2: 8.93%, old media rep2: 2.80%.

FIG. 12 shows violin plots from single cell RNA-seq of BHTP (new media) showing expression of FGF receptors in different cell clusters. FGFR1 is the predominant FGF receptor expressed in BHTP. FGFR1 is expressed in fibroblasts/stromal cells, cardiomyocytes, endothelial cells and cardiac progenitor cells.

FIG. 13 shows violin plots from single cell RNA-seq of BHTP (new media) showing expression of PDGF receptors in different cell clusters. PDGFRB is the predominant PDGF receptor expressed in BHTP. PDGFRB is predominantly expressed in the fibroblast/stromal population.

FIG. 14 shows new Serum Free (SF) human cardiac tissues are resistant to hypoxia. A) Schematic of the hypoxia testing. B) Staining after hypoxia in control and SF human cardiac tissue. C) Intensity of nuclei staining is higher in SF human cardiac tissue. D) Intensity of cardiomyocytes is unaltered in human cardiac tissue. E) Lactate dehydrogenase is lower in SF human cardiac tissue. F) Lactate is elevated in SF human cardiac tissue. G) The lactate to pyruvate ratio is highest in SF human cardiac tissue following hypoxia.

FIG. 15 shows implantation of the cardiac tissue patch in vivo. A) The patch was sutured onto the right ventricle of a 6 month old sheep heart. B) Image showing 2 BHTPs sutured onto the right ventricle of a 6 month-old sheep heart demonstrating near complete coverage of the entire surface of the right ventricle. C) Pericardium was closed with 3 stitches to help protect the patch. D)-F) Shows the patch was intact in situ after an acute monitoring period of 2.5 h (D and E) and after removal (F).

FIG. 16 shows functional validation of the patch in vivo. Steady-state Pressure Volume (PV) loops are shown at pre-patch, and 15 min & 90 min after patch implantation.

FIG. 17 shows the highly linear RV preload recruitable stroke work-end-diastolic volume relations for: pre-patch, and 15 min & 90 min after patch implantation.

FIG. 18 shows changes in mean aortic blood pressure (BP, panel A), mean pulmonary arterial (PA) blood pressure (panel B), heart rate (panel C), right ventricular (RV) output (panel D), the maximal rate of change of RV blood pressure, an index of RV contractility (RV dP/dtmax, panel E) and RV preload recruitable stroke work index (PRSWI, panel F) before patch implantation (Pre) and at 15 minutely intervals after patch implantation. Significance: No immediate change occurred in any variable with patch implantation and, with the exception of minor increases in PA blood pressure (<1 mmHg) and heart rate (10 beats/minute), variables were unchanged over a subsequent observation period to 120 min after patch implantation.

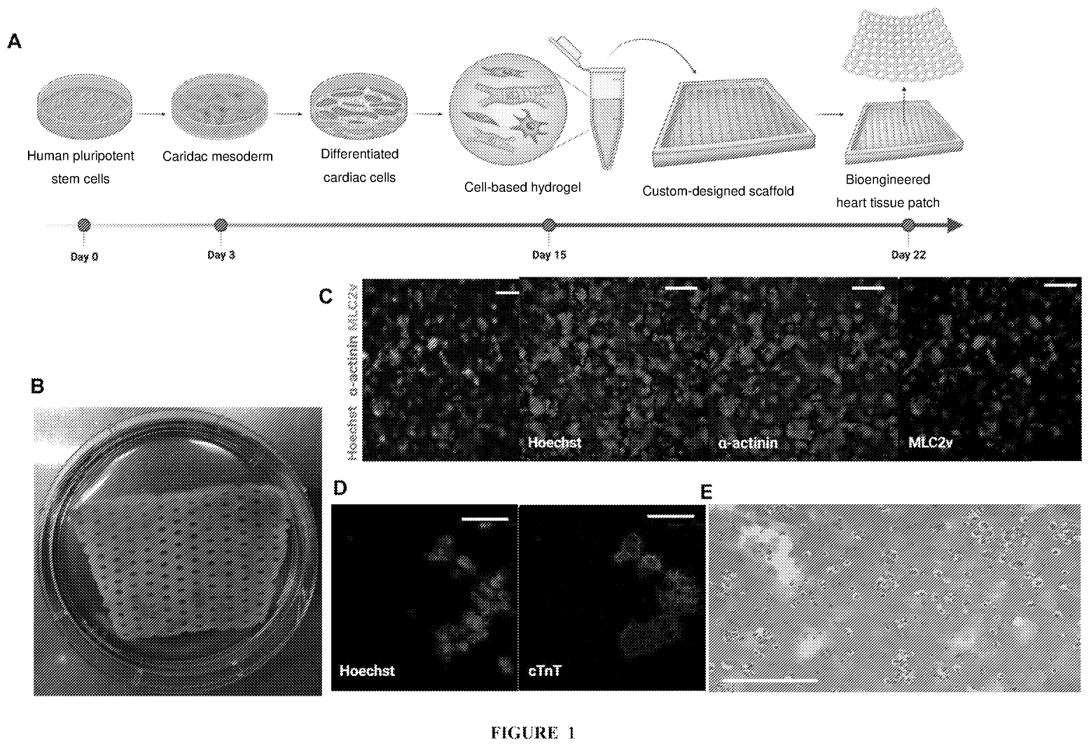

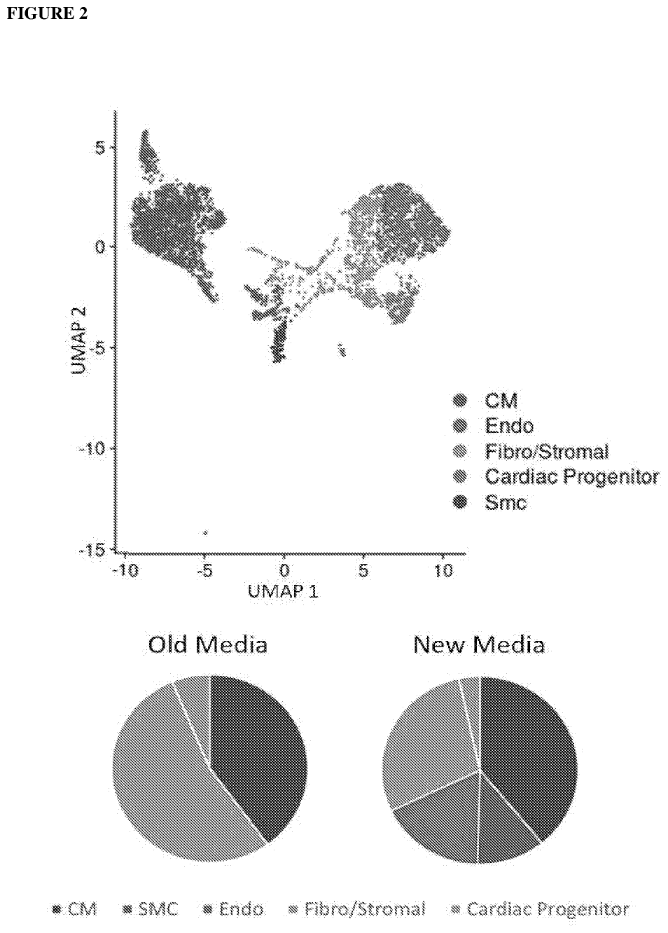

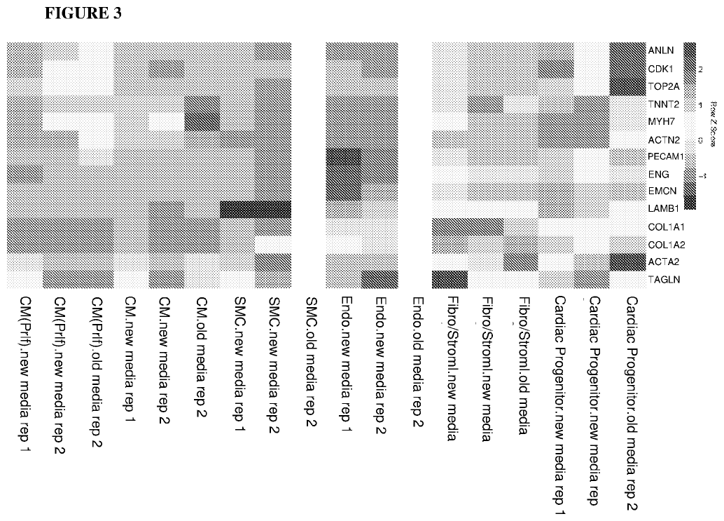

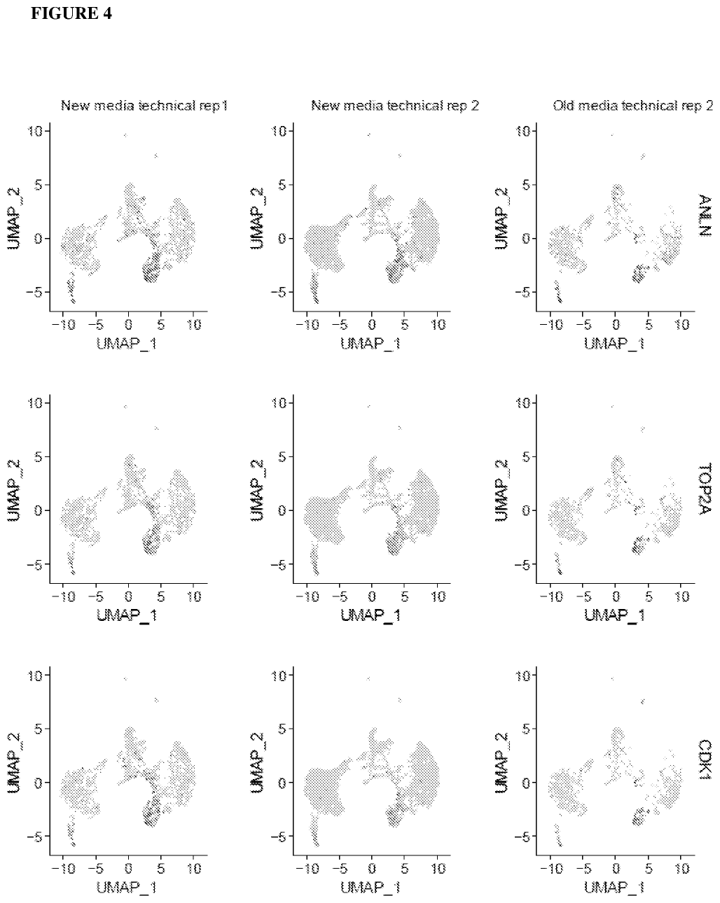

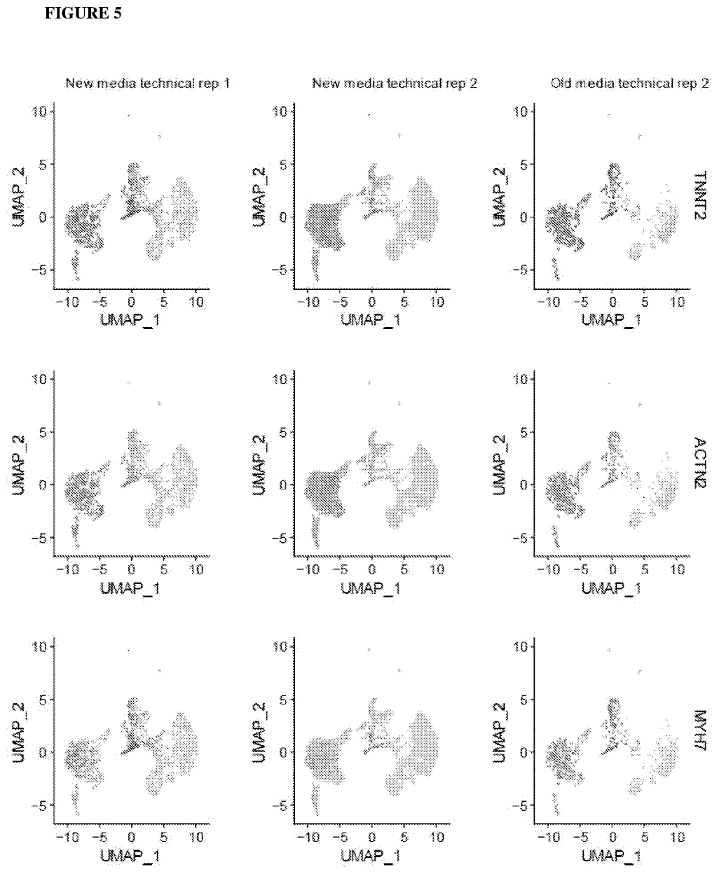

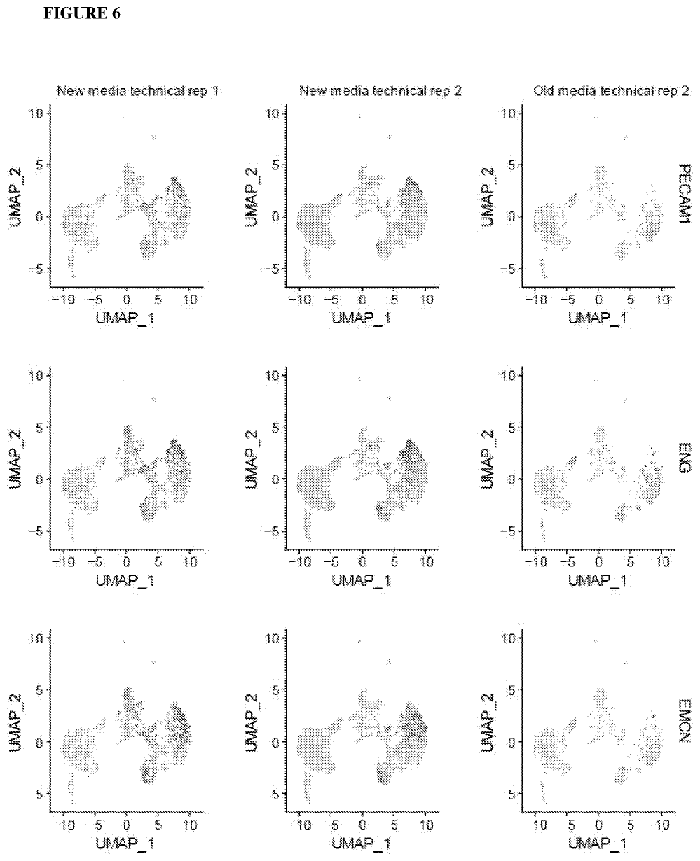

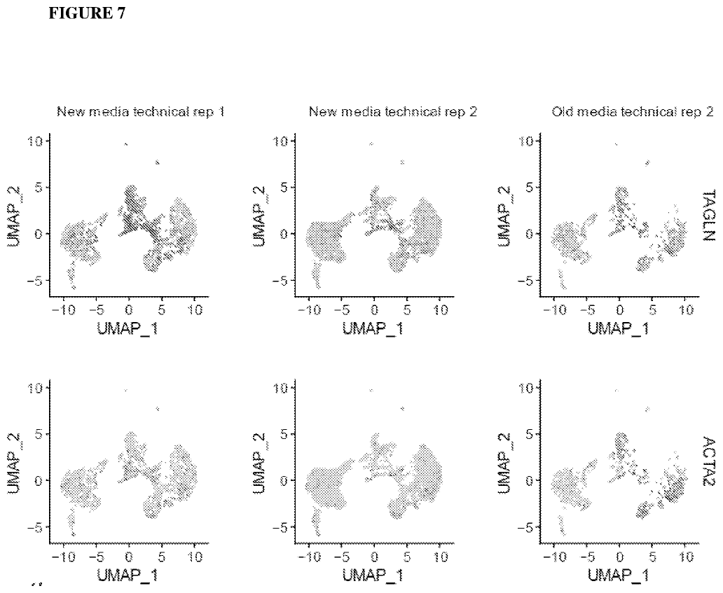

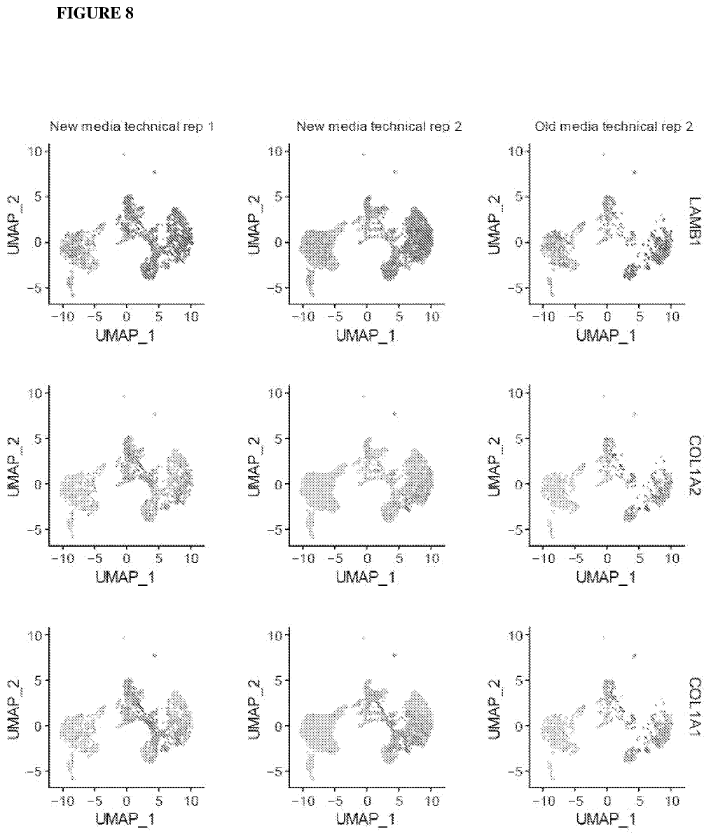

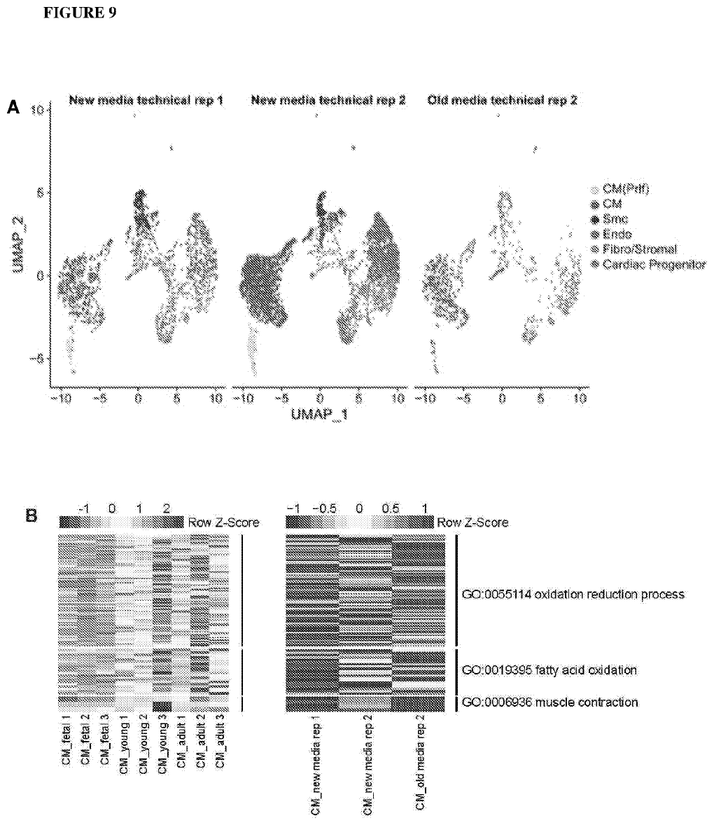

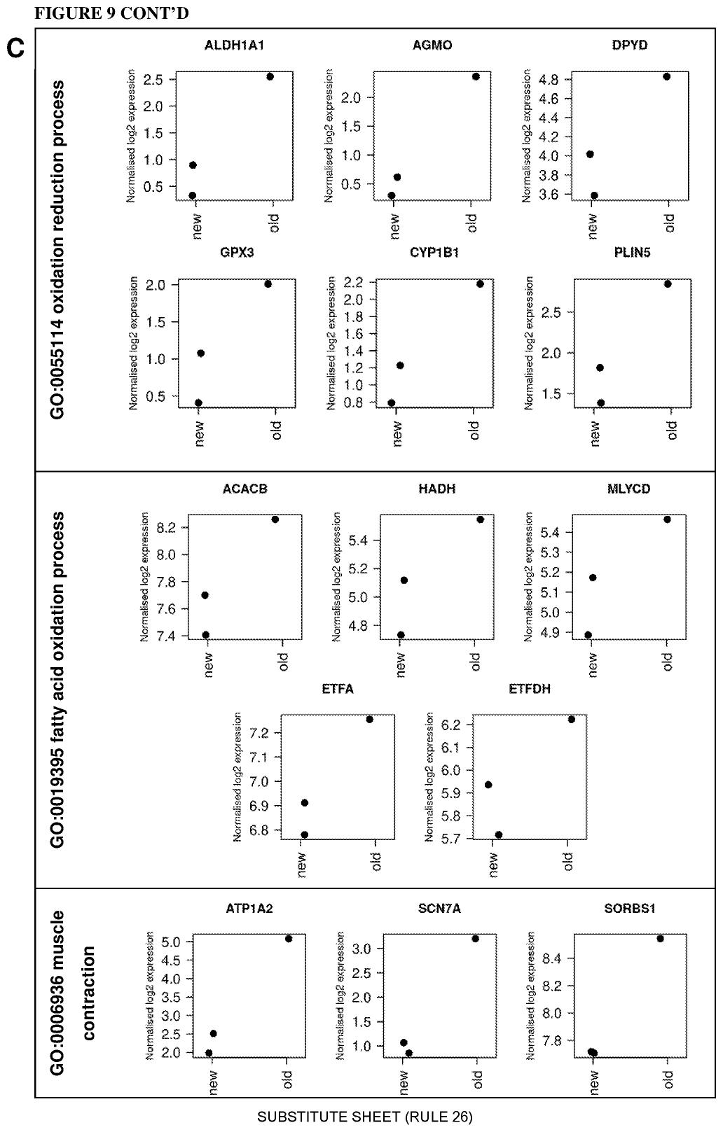

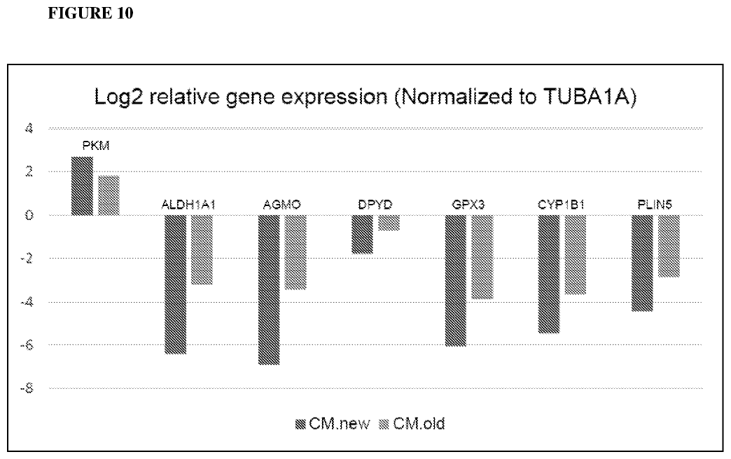

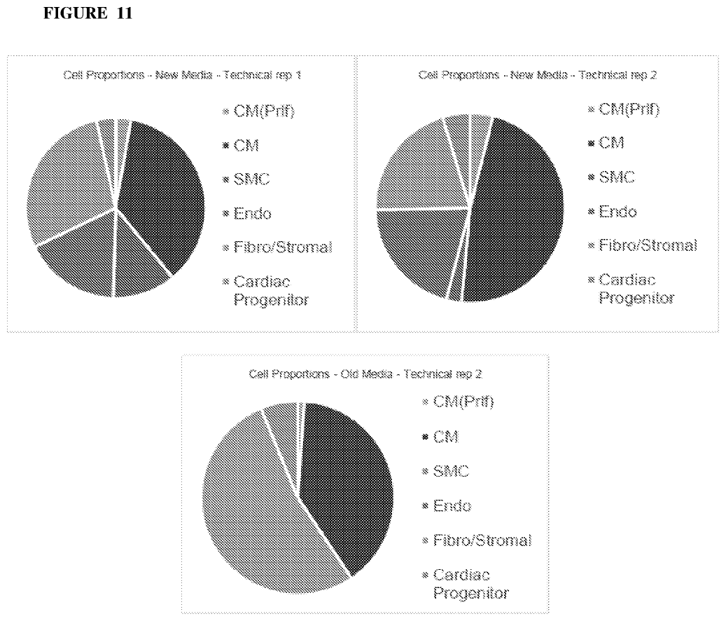

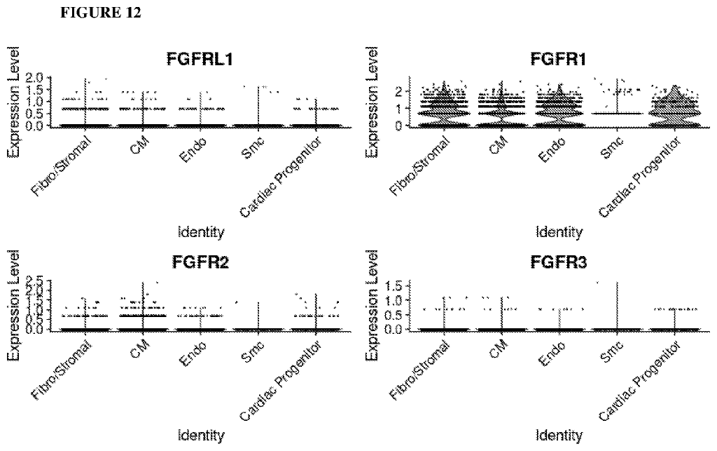

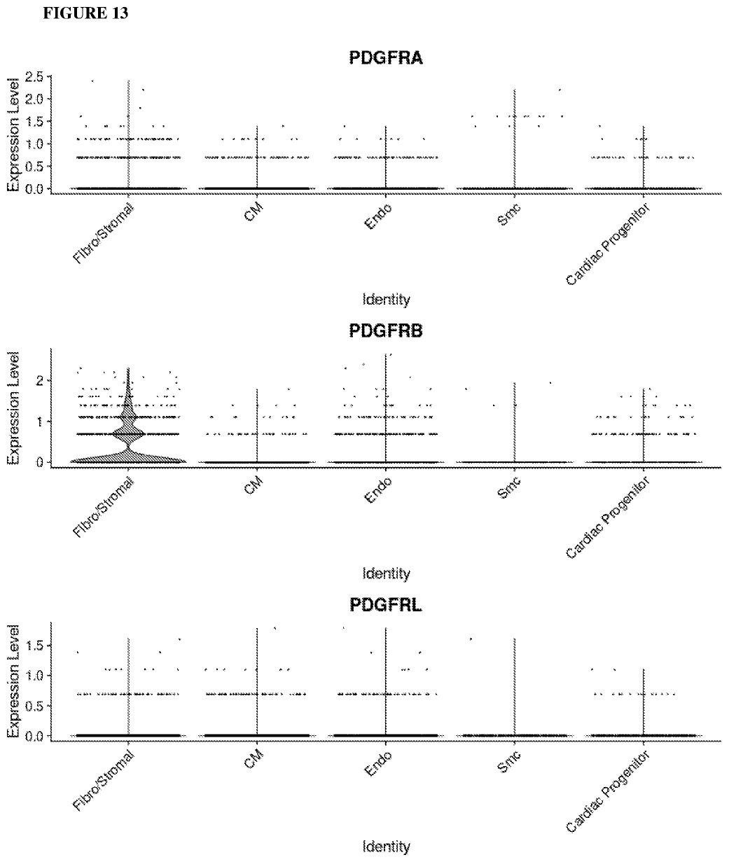

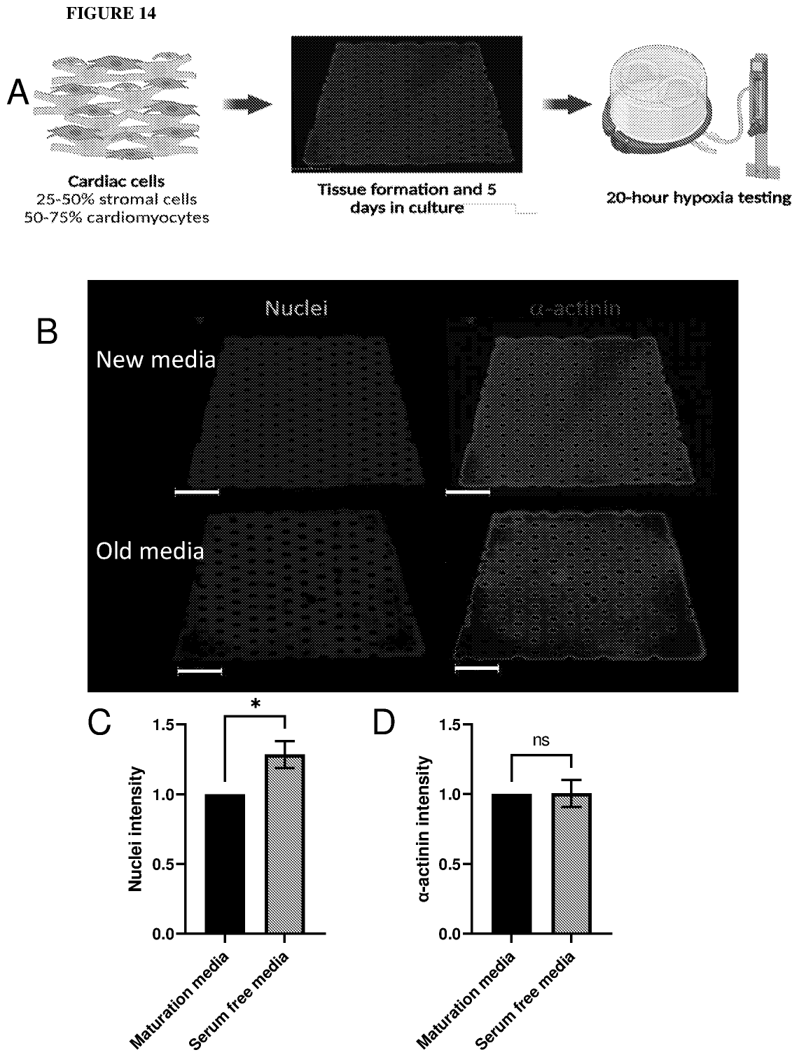

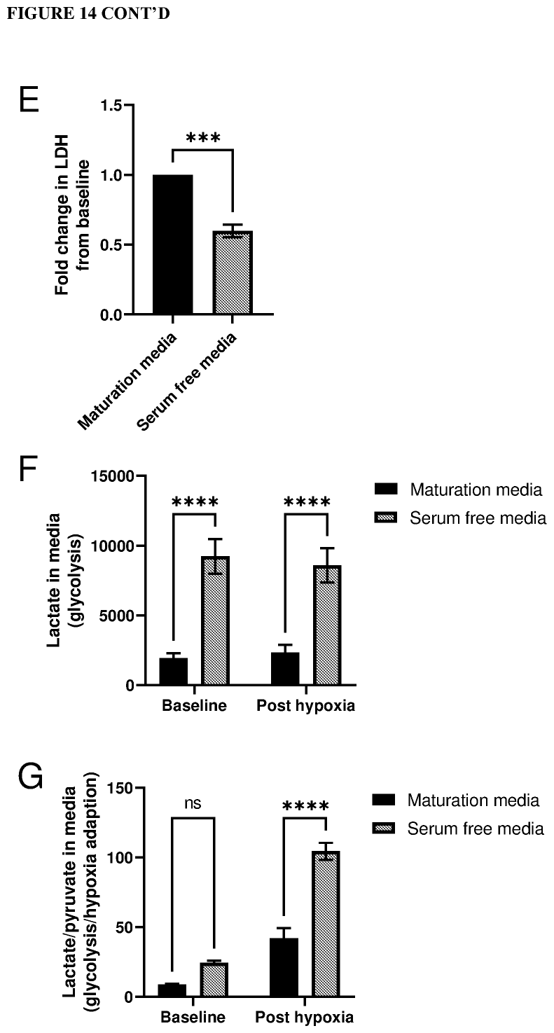

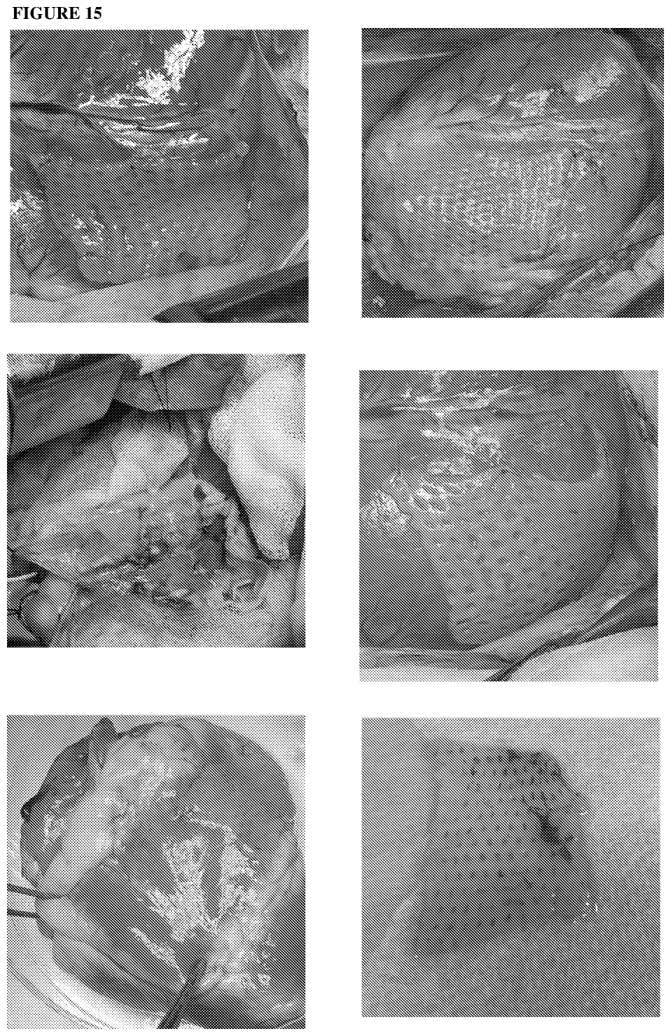

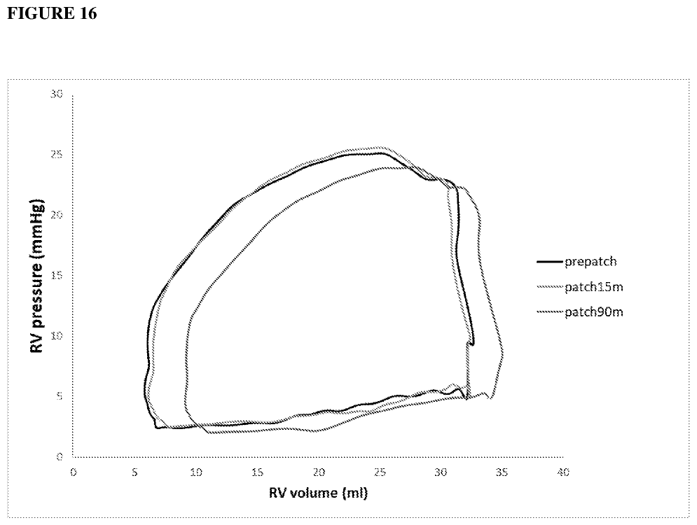

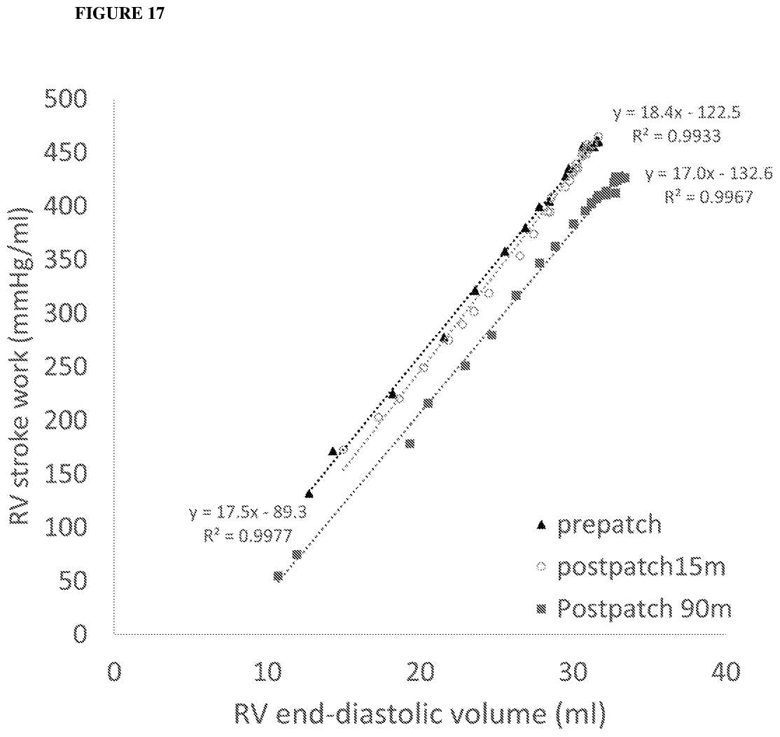

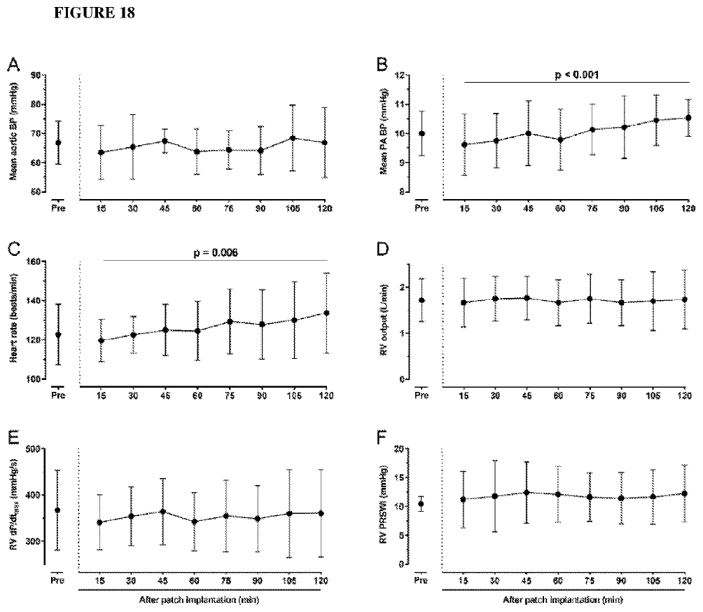

### DESCRIPTION OF EMBODIMENTS

### Definitions

Definitions of common terms in cellular and molecular biology, and biochemistry can be found in The Merck Manual of Diagnosis and Therapy, 20^{th} Edition, published by Merck Sharp & Dohme Corp., 2018 (ISBN 9780911910421, 0911910425); Robert S. Porter et al. (eds.), The Encyclopedia of Molecular Cell Biology and Molecular Medicine, published by Blackwell Science Ltd., 2008 (ISBN 3527305424, 9783527305421); and Robert A. Meyers (ed.), Molecular Biology and Biotechnology: a Comprehensive Desk Reference, published by VCH Publishers, Inc., 1995 (ISBN 1-56081-569-8); Immunology by Wemer Luttmann, published by Elsevier, 2006; Janeway's Immunobiology, Kenneth Murphy, Allan Mowat, Casey Weaver (eds.), Taylor & Francis Limited, 2016 (ISBN 9780815345510, 0815345518); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al, Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Laboratory Methods in Enzymology: RNA, Jon Lorsch (ed.) Elsevier, 2013 (ISBN: 9780124200371, 0124200370); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), Immunological Methods, Ivan Lefkovits, Benvenuto Pemis, (eds.) Elsevier Science, 2014 (ISBN: 9781483269993, 148326999X), the contents of which are all incorporated by reference herein in their entireties.

As used in this specification and the appended claims, terms in the singular and the singular forms “a,” “an” and “the,” for example, optionally include plural referents unless the content clearly dictates otherwise. For example, “a” cell includes one cell, one or more cells and a plurality of cells.

As used herein, the term “about”, unless stated to the contrary, refers to +/−10%, more preferably +/−5%, more preferably +/−1%, of the designated value. As used herein “up to about” refers to an amount of the recited entity which can be zero but does not exceed the designated value. For example, “up to about 10%” refers to an amount of 0% to about 10%.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not intended to limit the disclosure.

Throughout the specification, references to particular genes or proteins may be used interchangeably. A person skilled in the art will understand in the context whether the reference is intended to be a reference to the particular gene or the protein that is encoded by that gene.

The terms “human pluripotent stem cell” and “hPSC” refer to cells derived, obtainable or originating from human tissue that display pluripotency. The hPSC may be a human embryonic stem cell or a human induced pluripotent stem cell.

Human pluripotent stem cells may be derived from inner cell mass or reprogrammed using Yamanaka factors from many fetal or adult somatic cell types. The generation of hPSCs may be possible using somatic cell nuclear transfer.

The terms “human embryonic stem cell”, “hES cell” and “hESC” refer to cells derived, obtainable or originating from human embryos or blastocysts, which are self-renewing and pluri- or toti-potent, having the ability to yield all of the cell types present in a mature animal. Human embryonic stem cells (hESCs) can be isolated, for example, from human blastocysts obtained from human in vivo preimplantation embryos, in vitro fertilized embryos, or one-cell human embryos expanded to the blastocyst stage.

The terms “induced pluripotent stem cell” and “iPSC” refer to cells derivable, obtainable or originating from human adult somatic cells of any type reprogrammed to a pluripotent state through the expression of exogenous genes, such as transcription factors, including a preferred combination of OCT4, SOX2, KLF4 and c-MYC. hiPSC show levels of pluripotency equivalent to hESC but can be derived from a patient for autologous therapy with or without concurrent gene correction prior to differentiation and cell delivery.

More generally, the method disclosed herein could be applied to any pluripotent stem cell derived from any patient or a hPSC subsequently modified to generate a mutant model using gene-editing or a mutant hPSC corrected using gene-editing. Gene-editing could be by way of CRISPR, TALEN or ZF nuclease technologies.

As used herein, the term “cell culture” refers to any in vitro culture of cells. The term “culturing”refers to the process of growing and/or maintaining and/or manipulating a cell.

Included within this term are continuous cell lines (e.g., with an immortal phenotype), primary cell cultures, finite cell lines (e.g., non-transformed cells), and any other cell population maintained in vitro, including oocytes and embryos. As used herein, the terms “primary cell culture,” and “primary culture,” refer to cell cultures that have been directly obtained from cells in vivo, such as from a tissue specimen or biopsy from an animal or human. These cultures may be derived from adults as well as fetal tissue.

A “progenitor cell” is a cell which is capable of differentiating along one or a plurality of developmental pathways, with or without self-renewal. Typically, progenitor cells are unipotent or oligopotent and are capable of at least limited self-renewal.

The terms “differentiate”, “differentiating” and “differentiated”, relate to progression of a cell from an earlier or initial stage of a developmental pathway to a later or more mature stage of the developmental pathway. It will be appreciated that in this context “differentiated” does not mean or imply that the cell is fully differentiated and has lost pluripotency or capacity to further progress along the developmental pathway or along other developmental pathways. Differentiation may be accompanied by cell division.

As will be well understood in the art, the stage or state of differentiation of a cell may be characterized by the expression and/or non-expression of one of a plurality of markers. In this context, by “markers” is meant nucleic acids or proteins that are encoded by the genome of a cell, cell population, lineage, compartment or subset, whose expression or pattern of expression changes throughout development. Nucleic acid marker expression may be detected or measured by any technique known in the art including nucleic acid sequence amplification (e.g. polymerase chain reaction) and nucleic acid hybridization (e.g. microarrays, Northern hybridization, in situ hybridization), although without limitation thereto. Protein marker expression may be detected or measured by any technique known in the art including flow cytometry, immunohistochemistry, immunoblotting, protein arrays, protein profiling (e.g. 2D gel electrophoresis), although without limitation thereto.

Such terms are commonplace and well-understood by the skilled person when characterizing cell phenotypes. By means of additional guidance, when a cell is said to be positive for or to express or comprise expression of a given marker, such as a given gene or gene product, a skilled person would conclude the presence or evidence of a distinct signal for the marker when carrying out a measurement capable of detecting or quantifying the marker in or on the cell. Suitably, the presence or evidence of the distinct signal for the marker would be concluded based on a comparison of the measurement result obtained for the cell to a result of the same measurement carried out for a negative control (for example, a cell known to not express the marker) and/or a positive control (for example, a cell known to express the marker). Where the measurement method allows for a quantitative assessment of the marker, a positive cell may generate a signal for the marker that is at least 1.5-fold higher than a signal generated for the marker by a reference cell (e.g. negative control cell) or than an average signal generated for the marker by a population of reference or negative control cells, e.g., at least 2-fold, at least 4-fold, at least 10-fold, at least 20-fold, at least 30-fold, at least 40-fold, at least 50-fold higher, at least 100-fold higher, or even higher. Further, a positive cell may generate a signal for the marker that is 3.0 or more standard deviations, e.g., 3.5 or more, 4.0 or more, 4.5 or more, or 5.0 or more standard deviations, higher than an average signal generated for the marker by a population of reference or negative control cells.

As used herein, the terms “culture medium” and “cell culture medium” refer to media that are suitable to support the growth of cells in vitro (i.e., cell cultures, cell lines, etc.). It is not intended that the term be limited to any particular culture medium. For example, it is intended that the definition encompass maintenance media as well as other media for the differentiation or specialization of cells. Indeed, it is intended that the term encompass any culture medium suitable for the growth of the cell cultures and cells of interest.

As used herein, “tissue” means an aggregate of cells. In some embodiments, the cells in the tissue are cohered or fused.

The terms “individual”, “subject”, “host” and “patient” are used interchangeably herein and refer to any mammalian subject for whom diagnosis, treatment, or therapy is desired, particularly humans.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “decrease”, “reduced”, “reduction”, “to a lesser extent,” or “inhibit” are all used herein to mean a decrease or lessening of a property, level, or other parameter by a statistically significant amount. In some embodiments, “reduced”, “reduction”, “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “increases”, or “enhance” or “activate” or “to a greater extent” are all used herein to generally mean an increase of a property, level, or other parameter by a statistically significant amount; for the avoidance of any doubt, the terms “increased”, “increase”, “to a greater extent,” “enhance” or “activate” can refer to an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, a “reference level” refers to the level of a marker or parameter in a normal, otherwise unaffected cell population or tissue (e.g., a cell, tissue, or biological sample obtained from a healthy subject, or a biological sample obtained from the subject at a prior time point, e.g., cell, tissue, or a biological sample obtained from a patient prior to being diagnosed with a disease, or a biological sample that has not been contacted with an agent or composition as disclosed herein). Alternatively, a reference level can also refer to the level of a given marker or parameter in a subject, organ, tissue, or cell, prior to administration of a treatment, e.g., with an agent or via administration of a transplant composition.

As used herein, a “control” or an “appropriate control” refers to an untreated, otherwise identical cell, subject, organism, or population (e.g., a cell, tissue, or biological sample that was not contacted by an agent or composition described herein) relative to a cell, tissue, biological sample, or population contacted or treated with a given treatment. For example, an appropriate control can be a cell, tissue, organ or subject that has not been contacted with an agent or administered a cell as described herein. In one example, the control is a cell, tissue, biological sample, or population cultured in a maturation medium as described in the examples herein.

In one or more embodiments described herein, assessing the expression of various genes includes comparing the fold change. In one embodiment, the fold change is used to measure the change in the expression level of genes. In one embodiment the expression of a gene can be expressed as relative expression comparative to a housekeeping gene. In one embodiment, the fold change is measured by RPKM. As used herein, the term “RPKM” refers to “Reads Per Kilobase per Million mapped reads”. The term RPKM refers to a method of quantifying gene expression from RNA sequencing data by normalizing for total read length and the number of sequencing reads. In one embodiment, RPKM calculation provides a normalization for comparing gene coverage values. The RPKM value corrects for differences in both sample sequencing depth and gene length. In one example, the RPKM can be calculated via the following formula: numReads/(geneLength/1000*totalNumReads/1,000,000) ; wherein, “numReads” refers to the number of reads mapped to a gene sequence; wherein, “geneLength” refers to the length of the gene sequence; and wherein, “totalNumReads” refers to the total number of mapped reads of a sample.

The term “agonist” or “activator” may be used interchangeably and as used herein means an activator, for example, of a pathway or signaling molecule. An agonist of a molecule can retain substantially the same, or a subset, of the biological activities of the molecule (e.g. FGF). For example, an FGF agonist or FGF activator means a molecule that selectively activates FGF signaling.

The term “inhibitor” as used herein means a selective inhibitor, for example of a pathway or signaling molecule. An inhibitor or antagonist of a molecule can inhibit one or more of the activities of the naturally occurring form of the molecule.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment” or “an example embodiment” means that a particular feature, structure or characteristic described in connection with the embodiment is included in at least one embodiment of the present invention. Thus, appearances of the phrases “in one embodiment”, “in an embodiment” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination. Any example or embodiment herein shall be taken to apply mutatis mutandis to any other example or embodiment unless specifically stated otherwise.

The present disclosure is not to be limited in scope by the specific examples described herein, which are intended for the purpose of exemplification only. Functionally-equivalent methods and systems are clearly within the scope of the disclosure, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The disclosure is hereinafter described by way of the following non-limiting Examples and with reference to the accompanying drawings. Although the examples herein concern humans and the language is primarily directed to human concerns, the concepts described herein are applicable to other animals. These and other aspects and features of the present disclosure will become more fully apparent from the following description and appended claims, or may be learned by the practice of the disclosure as set forth hereinafter.

A reference herein to a patent document or other matter which is given as prior art is not to be taken as an admission that that document or matter was known or that the information it contains was part of the common general knowledge as at the priority date of any of the claims.

### Methods for the Generation of Engineered Human Cardiac Tissue

The present invention provides a method for producing engineered human cardiac tissue comprising endothelial cells, the method comprising culturing a cell suspension comprising a population of human pluripotent stem cell (hPSC) culture-derived cardiomyocytes and non-cardiomyocyte cells in the presence of a FGFR1 agonist and a PDGFR agonist for a time and under conditions sufficient to produce engineered human cardiac tissue comprising endothelial cells.

The present invention also provides a method for producing engineered human cardiac tissue, the method comprising the steps of: i) mixing a cell suspension comprising a population of human pluripotent stem cell (hPSC) culture-derived cardiomyocytes and non-cardiomyocyte cells with a flowable hydrogel composition, wherein said cell suspension is in a serum-free medium; ii) loading the flowable hydrogel composition comprising said cell suspension into a mold and incubating the hydrogel for a time and under conditions sufficient to solidify the hydrogel; and iii) culturing the hydrogel from step ii); wherein the population of human pluripotent stem cell (hPSC) culture-derived cardiomyocytes and non-cardiomyocyte cells is contacted with an FGFR1 agonist and a PDGFR agonist to thereby provide engineered human cardiac tissue, wherein said tissue comprises cardiomyocytes, stromal cells/fibroblasts and endothelial cells.

In one embodiment, prior to mixing in step i), the FGFR1 agonist and/or the PDGFR agonist are added to the cell suspension in serum free medium or the flowable hydrogel composition. In another embodiment, prior to mixing in step i), the population of human pluripotent stem cell (hPSC) culture-derived cardiomyocytes and non-cardiomyocyte cells are cultured in a serum free medium comprising a FGFR1 agonist and a PDGFR agonist. In an alternative embodiment, the FGFR1 agonist and/or the PDGFR agonist are added to the flowable hydrogel composition comprising said cell suspension after said mixing in step i). In another embodiment, the solidified hydrogel of step ii) is cultured in step iii) in a serum free medium comprising the FGFR1 agonist and the PDGFR agonist.

The present invention provides a method for producing engineered human cardiac tissue, the method comprising the steps of: i) mixing a cell suspension comprising a population of human pluripotent stem cell (hPSC) culture-derived cardiomyocytes and non-cardiomyocyte cells with a flowable hydrogel composition, wherein said cell suspension is in a serum-free medium comprising a FGFR1 agonist and a PDGFR agonist; ii) loading the flowable hydrogel composition comprising said cell suspension into a mold and incubating the hydrogel for a time and under conditions sufficient to solidify the hydrogel; and iii) culturing the hydrogel from step ii) in serum free medium as defined in step i); to thereby provide engineered human cardiac tissue, wherein said tissue comprises cardiomyocytes, stromal cells/fibroblasts and endothelial cells. In a further embodiment, the FGFR1 agonist and/or the PDGFR agonist are also added to the flowable hydrogel composition prior to mixing in step i). In a further embodiment, the FGFR1 agonist and/or the PDGFR agonist are also added to the flowable hydrogel composition comprising said cell suspension after said mixing in step ii). In a further embodiment, the solidified hydrogel of step ii) is cultured in step iii) in a serum free medium comprising the FGFR1 agonist and the PDGFR agonist.

In one embodiment the suspension of cells comprises about 50% to about 80% cardiomyocytes and about 20% to about 50% non-cardiomyocyte cells. In a preferred embodiment the suspension comprises about 75%, about 70%, about 65%, about 60%, about 55% or about 50% cardiomyocytes and about 25%, about 30%, about 35%, about 40%, about 45%, or about 50% non-cardiomyocytes. In a particularly preferred embodiment, the suspension of cells comprises about 70% cardiomyocytes and about 30% non-cardiomyocyte cells. In another embodiment, the suspension of cells comprises less than 5% CD31+ endothelial cells. In another embodiment the suspension of cells comprises about 50% to about 80% cardiomyocytes and about 20% to about 50% stromal cells. In a preferred embodiment the suspension comprises about 50 to about 75%, about 50 to about 70%, about 60 to about 70%, about 50 to about 65%, about 60% to about 80%, about 55% to about 75%, about 55% to about 80% or about 65 to about 80% cardiomyocytes and about 25% to about 50%, about 25% to about 45%, about 30% to about 50%, about 25% to about 50%, about 35% to about 50%, about 25% to about 40%, about 25% to about 45%, about 20 to about 45% or about 20 to about 40% stromal cells. In a preferred embodiment the suspension comprises about 75%, about 70%, about 65%, about 60%, about 55% or about 50% cardiomyocytes and about 25%, about 30%, about 35%, about 40%, about 45%, or about 50% stromal cells. In a particularly preferred embodiment, the suspension of cells comprises about 70% cardiomyocytes and about 30% stromal cells. In a preferred embodiment, the cardiomyocytes are actinin⁺/CTNT⁺ cardiomyocytes, and the non-cardiomyocytes or stromal cells are CD90⁺ stromal cells. In some embodiments, suspension of cells consists essentially of a population of human pluripotent stem cell (hPSC) culture-derived cardiomyocytes and stromal cells. In one embodiment, the suspension of cells consisting essentially of a population of human pluripotent stem cell (hPSC) culture-derived cardiomyocytes and stromal cells comprises about 50% to about 80% cardiomyocytes and about 20% to about 50% stromal cells or any of the other ranges of cardiomyocytes and and stromal cells described above.

In a further preferred embodiment, the cell suspension which comprises cardiomyocytes and non-cardiomyocytes which are derived from hPSC cell culture are obtained from a single differentiation process. That is, it will be understood that a starting population of hPSCs may, through the directed differentiation towards cardiac lineage, give rise to both the cardiomyocytes and non-cardiomyocytes such that for the further culture of the cell suspension and the generation of cardiac tissue comprising cardiomyocytes, stromal cells/fibroblasts and endothelial cells, it is not necessary to combine, mix or seed the cell suspension with another population of cells, such as endothelial cells, for example.

As described herein, it is the surprising finding of the inventors that through the culture of a suspension of cells comprising cardiomyocytes and non-cardiomyocytes in the presence of the combination of an FGFR1 agonist and a PDGFR agonist, human engineered cardiac tissue can be generated wherein the tissue more appropriately reflects the heterogeneity of cell types present in native human cardiac tissue. That is, the human engineered cardiac tissue comprises cardiomyocytes, stromal cells/fibroblasts and endothelial cells. In some embodiments, the engineered cardiac tissue may also further comprise vascular smooth muscle cells and/or cardiac progenitor cells. In some embodiments, the human engineered cardiac tissue comprises cardiomyocytes, stromal cells/fibroblasts, endothelial cells and vascular smooth muscle cells.

In one aspect, the present invention provides a method for producing a population of human pluripotent stem cell (hPSC) culture-derived cardiomyocytes, stromal cells/fibroblasts and endothelial cells, the method comprising culturing a cell suspension comprising a population of human pluripotent stem cell (hPSC) culture-derived cardiomyocytes and non-cardiomyocyte cells in the presence of a FGFR1 agonist and a PDGFR agonist for a time and under conditions sufficient to produce a population of human pluripotent stem cell (hPSC) culture-derived endothelial cells. In another aspect, the present invention provides the use of a FGFR1 agonist and a PDGFR agonist to produce a population of human pluripotent stem cell (hPSC) culture-derived endothelial cells in a cell suspension comprising a population of human pluripotent stem cell (hPSC) culture-derived cardiomyocytes and non-cardiomyocyte cells. As described above, in some embodiments the suspension of cells comprises about 50% to about 80% cardiomyocytes and about 20% to about 50% non-cardiomyocyte cells, or any of the other ranges of cardiomyocytes and non-cardiomyocytes described above. In another embodiment, the suspension of cells comprises less than 5% CD31+ endothelial cells. As described above, in some embodiments the suspension of cells comprises about 50% to about 80% cardiomyocytes and about 20% to about 50% stromal cells or any of the other ranges of cardiomyocytes and and stromal cells described above. In a preferred embodiment, the cardiomyocytes are actinin+/CTNT+ cardiomyocytes, and the non-cardiomyocytes or stromal cells are CD90+ stromal cells. In some embodiments, suspension of cells consists essentially of a population of human pluripotent stem cell (hPSC) culture-derived cardiomyocytes and stromal cells. In one embodiment, the suspension of cells consisting essentially of a population of human pluripotent stem cell (hPSC) culture-derived cardiomyocytes and stromal cells comprises about 50% to about 80% cardiomyocytes and about 20% to about 50% stromal cells or any of the other ranges of cardiomyocytes and and stromal cells described above.

In one embodiment, the engineered cardiac tissue comprises about 30% to about 70% cardiomyocytes; about 10% to about 35% stromal cells/fibroblasts; and about 2% to about 30% endothelial cells. In another embodiment, the engineered cardiac tissue comprises about 30% to about 70% cardiomyocytes; about 15% to about 35% stromal cells/fibroblasts; and about 5% to about 30% endothelial cells. In another embodiment, the engineered cardiac tissue comprises about 30% to about 65% cardiomyocytes; about 15% to about 35% stromal cells/fibroblasts; and about 5% to about 30% endothelial cells. In another embodiment, the engineered cardiac tissue comprises about 30% to about 60% cardiomyocytes; about 15% to about 35% stromal cells/fibroblasts; and about 5% to about 30% endothelial cells. In another embodiment, the engineered cardiac tissue comprises about 30% to about 55% cardiomyocytes; about 15% to about 35% stromal cells/fibroblasts; and about 5% to about 30% endothelial cells. In another embodiment, the engineered cardiac tissue comprises about 30% to about 55% cardiomyocytes; about 20% to about 35% stromal cells/fibroblasts; and about 15% to about 30% endothelial cells. In a preferred embodiment, the engineered cardiac tissue comprises at least about 30% cardiomyocytes; about 35% or less stromal cells/fibroblasts; and about 30% or less endothelial cells. In some embodiments, the cardiac tissue further comprises up to about 15% vascular smooth muscle cells. In some embodiments, the cardiac tissue further comprises up to about 10% cardiac progenitor cells. In a more preferred embodiment, the engineered cardiac tissue comprises about 30% to about 50% cardiomyocytes; about 20% to about 35% stromal cells/fibroblasts; and about 15% to about 30% endothelial cells. In a more preferred embodiment, the engineered cardiac tissue comprises about 30% to about 55% cardiomyocytes; about 20% to about 35% stromal cells/fibroblasts; about 5% to about 30% endothelial cells; up to about 15% vascular smooth muscle cells; and up to about 10 % cardiac progenitor cells. In a more preferred embodiment, the engineered cardiac tissue comprises about 30% to about 55% cardiomyocytes; about 20% to about 35% stromal cells/fibroblasts; about 5% to about 30% endothelial cells; about 0.5% to about 15% vascular smooth muscle cells; and about 0.5% to about 10 % cardiac progenitor cells. In a more preferred embodiment, the engineered cardiac tissue comprises about 30% to about 55% cardiomyocytes; about 20% to about 35% stromal cells/fibroblasts; about 15% to about 30% endothelial cells; about 0.5% to about 15% vascular smooth muscle cells; and about 0.5% to about 10 % cardiac progenitor cells. In some embodiments, the engineered cardiac tissue comprises about 30% to about 45% cardiomyocytes, about 30% to about 35% stromal cells/fibroblasts; and about 20% to about 24% endothelial cells. In some embodiments, the engineered cardiac tissue comprises about 30% to about 45% cardiomyocytes, about 20% to about 30% stromal cells/fibroblasts; and about 15% to about 30% endothelial cells. In some embodiments, the engineered cardiac tissue comprises about 30% to about 45% cardiomyocytes, about 30% to about 35% stromal cells/fibroblasts; and about 26% to about 30% endothelial cells. In some embodiments, the engineered cardiac tissue comprises about 30% to about 45% cardiomyocytes, about 20% to about 30% stromal cells/fibroblasts; and about 15% to about 30% endothelial cells. In some embodiments, the engineered cardiac tissue comprises about 30% to about 45% cardiomyocytes, about 30% to about 35% stromal cells/fibroblasts; and about 20% endothelial cells. In some embodiments, the engineered cardiac tissue comprises about 30% to about 45% cardiomyocytes, about 20% to about 30% stromal cells/fibroblasts; and about 15% to about 25% endothelial cells. In some embodiments, the engineered cardiac tissue comprises about 30% to about 45% cardiomyocytes, about 30% to about 35% stromal cells/fibroblasts; and about 30% endothelial cells. In some embodiments, the engineered cardiac tissue comprises about 30% to about 45% cardiomyocytes, about 20% to about 30% stromal cells/fibroblasts; and about 15% to about 25% endothelial cells.

In some embodiments, the engineered cardiac tissue comprises about 2% to about 5% proliferative cardiomyocytes. In a further embodiment, at least about 3% to about 10% of the cardiomyocytes of the engineered cardiac tissue are proliferative as determined by the expression of one or more markers associated with cell proliferation. In a further embodiment, about 3%, about 4%, about 5%, about 6%, about 7%, about 8%, about 9%, or about 10% of the cardiomyocytes of the engineered cardiac tissue are proliferative as determined by the expression of one or more markers associated with cell proliferation. In one embodiment, the proliferative cardiomyocytes express one or more markers selected from the group consisting of ANLN, TOP2A, CDK1, and E2F1.

Another characteristic of the cardiomyocytes present in the engineered cardiac tissues is that they are MMP1⁺. In a preferred embodiment therefore, the cardiomyocytes in the engineered cardiac tissue are MMP1⁺.

Another characteristic of the fibroblasts present in the engineered cardiac tissues is that they are MMP1⁺, EMP1⁺, FOXD1⁺, RAB27B⁺, NR2F1⁺, F2RL1⁺, SPP1⁺, TMEM158⁺, PTHLH⁺, PHLDA2⁺, MALL⁺, MYCT1⁺, DUSP4⁺, PLAU⁺, TMEM156⁺, CD274⁺, MMP10⁺, ARRDC4⁺, RFX8⁺, MLPH⁺, THBD⁺, HHEX⁺, VGF⁺, OTULINL⁺, IL33⁺, CA12⁺, C6orf141⁺, MFSD2A⁺ and/or CARD10⁺. In a preferred embodiment therefore, the fibroblasts in the engineered cardiac tissue are positive for one or more of these aforementioned markers.

As set out herein, the inventors have identified that FGFR1 is the predominant FGF receptor expressed in the tissue engineered cardiac tissue according to methods of the invention. Accordingly, in a preferred embodiment, the method of the invention comprises culturing the cells in a basal medium which is supplemented with an FGFR1 agonist selected from the group consisting of FGF1, FGF2, FGF3, FGF4, FGF5, FGF6, FGF7, FGF8, FGF9, FGF10, FGF16, FGF17, FGF18, FGF19, FGF20, FGF21, FGF22, and FGF23. In some embodiments, the method of the invention comprises culturing the cells in a basal medium which is supplemented with an FGFR1 agonist selected from the group consisting of FGF1, FGF2, FGF3, FGF4, FGF5, FGF6, FGF8, FGF9, FGF16, FGF17, FGF18, FGF19, FGF20,FGF21, FGF22, and FGF23. In some embodiments, the method of the invention comprises culturing the cells in a basal medium which is supplemented with an FGFR1 agonist that is a member of the FGF1 subfamily, the FGF4 subfamily, the FGF8 subfamily, the FGF9 subfamily or the FGF19 subfamily. In some embodiments, the method of the invention comprises culturing the cells in a basal medium which is supplemented with an FGFR1 agonist selected from the group consisting of FGF1, FGF2, FGF4, FGF5, FGF6, FGF9, FGF16 and FGF20. More preferably, the FGFR1 agonist is FGF2. In one embodiment, the medium comprises about 0.5-100 ng/ml FGF2. Preferably, the concentration of FGF2 is about 1-50 ng/ml, more preferably about 5-20 ng/ml and most preferably about 10 ng/mL.

As set out herein, the inventors have also identified that PDGFRβ is the predominant PDGF receptor expressed in the tissue engineered cardiac tissue according to methods of the invention. Accordingly, in a preferred embodiment, the method of the invention comprises culturing the cells in a basal medium which is supplemented with an PDGFR agonist selected from the group consisting of PDGF-AA, PDGF-BB, PDGF-AB, PDGF-CC or PDGF-DD. More preferably, the PDGFR1 agonist is PDGF-BB. In one embodiment, the medium comprises about 0.5-100 ng/ml PDGF-BB. Preferably, the concentration of PDGF-BB is about 1-50 ng/ml, more preferably about 5-20 ng/mL and most preferably about 10 ng/mL.

In one embodiment, the serum free medium further comprises a basal medium comprising albumin and transferrin. In yet a further preferred embodiment, the basal medium comprises a commercially available supplement comprising albumin and transferrin. In a preferred embodiment, such a supplement is a B27® supplement or B27® supplement minus insulin. In a preferred embodiment, the B27® supplement or B27® supplement minus insulin is applied in an amount of 0.1-10 % B27® or B27® minus insulin, preferably 0.5-8 %, more preferably 1-6 %, even more preferably 1.5-4%, and most preferably about 4% B27® or B27® minus insulin. In one embodiment, B27 supplement comprises Biotin, DL Alpha Tocopherol Acetate, DL Alpha-Tocopherol, Vitamin A (acetate), BSA, fatty acid free Fraction V, Catalase, Human Recombinant Insulin, Human Transferrin, Superoxide Dismutase, Corticosterone, D-Galactose, Ethanolamine HCl, Glutathione (reduced), L-Carnitine HCl, Linoleic Acid, Linolenic Acid, Progesterone, Putrescine 2HCl, Sodium Selenite, and T3 (triodo-I-thyronine).

Through detailed studies the inventors have determined that culturing the cell seeded hydrogels in a culture medium which has a low level of calcium is of importance for preserving a phenotype in the engineered cardiac tissue which demonstrates glycolytic metabolism and reduced contractility. Accordingly, in one embodiment, the basal medium is a low calcium medium having a calcium concentration that is less than the physiological calcium concentration, for example, less than about 2 mM. In another embodiment, the basal medium is a low calcium medium having a calcium concentration of less than about 1.2 mM. In yet another embodiment, basal medium is a low calcium medium having a calcium concentration of less than about 1 mM. In a further preferred embodiment, the basal medium is a low calcium medium having a calcium concentration of less than about 0.8 mM, more preferably less than 0.75 mM, even more preferably less than about 0.5 mM, most preferably about 0.43 mM. In some embodiments, the basal medium is a low calcium medium having a calcium concentration of from about 0.2 mM to about 1 mM. The skilled person will appreciate that any suitable basal medium having the aforementioned preferred calcium concentrations may be used in the methods such as alphaMEM, DMEM or RPMI. In another preferred embodiment the basal medium is RPMI medium.

Usually, the culturing step is carried out for about 5 days to about 7 days. Preferably, the culturing step is carried out for about 7 days. In a further preferred embodiment, the method comprises replacing the medium in the culturing step for fresh medium comprising an FGFR1 agonist and a PDGFR agonist at least every 2 days.

In one aspect there is provided a media composition suitable for producing a population of human pluripotent stem cell (hPSC) culture-derived cardiomyocytes, stromal cells/fibroblasts and endothelial cells comprising a basal medium having a calcium concentration that is less than the physiological calcium concentration, an FGFR1 agonist and a PDGFR agonist. In some embodiments, the media composition may also include a commercially available supplement comprising albumin and transferrin. Amounts and concentrations of each of the components of the media composition are as described herein.

As demonstrated in the examples below, it has been proven advantageous that the engineered cardiac tissues generated by methods of the present invention and in the engineered cardiac tissue of the present invention as described below, display glycolytic metabolism. This may be advantageous as it provides resistance to hypoxia induced cell death in comparison to cells using oxidative phosphorylation. As implantation in vivo will lead to a period of hypoxia this may lead to better survival. Additionally, glycolysis supports cardiomyocyte proliferation (Mills et al. PNAS 2017) which may be advantageous to promote expansion of cell numbers following implantation.

In one embodiment, the cardiac tissues generated using the methods and the engineered cardiac tissue described herein display log2 relative gene expression >2 for PKM (normalized to TUBA1A). Another characteristic of the cardiac tissues generated using the methods described herein is that after 24 h in SF culture medium, the culture medium has a concentration of lactate of at least 0.5 mM or at least 1 mM. In another embodiment, after culture of the cardiac tissues generated using the methods described herein for 24 h in SF culture medium, the culture medium has a concentration of lactate of about 10-20 nmol/million cells/h.

As demonstrated in the examples below, it has been proven advantageous that the engineered cardiac tissues generated by methods of the present invention and the engineered cardiac tissue of the present invention display tolerance to hypoxia. In one embodiment, the cardiac tissues generated using the methods and the engineered cardiac tissue described herein display log2 relative gene expression of <−4 for ALDH1, AGMO, GPX3, CYP1B1 and PLIN5 (normalized to TUBA1A). Another characteristic of the cardiac tissues generated using the methods described herein and the engineered cardiac tissue itself (described below) is that after 20 h of culture under hypoxic conditions, cell culture medium from such culture has a lactate/pyruvate ratio of at least 1:1. Yet another characteristic of the cardiac tissues generated using the methods described herein and the engineered cardiac tissue itself (described below) is that after 20 h of culture under hypoxic conditions, cell death is at least 1-fold lower compared to control cadiac tissue, and cell death may be at least 2-fold, at least 3-fold or more lower compared to control cadiac tissue. Cell death may be assessed using routine methods known to the skilled addressee, including but not limited to LDH assays. In a preferred embodiment, after 20 h of culture of the cardiac tissues generated using the methods described herein and the engineered cardiac tissue itself (described below) under hypoxic conditions, cell death is at least 1-fold, at least 2-fold, at least 3-fold or more lower compared to control cadiac tissue, as measured by an LDH assay.

As demonstrated in the examples below, it has been proven advantageous that the engineered cardiac tissues generated by methods of the present invention display low contractility. Contractility of the engineered tissue may be assessed and measured using techniques known to the skilled person (see e.g. Tibucy et al. Circulation. 2017; 135(19): 1832-1847 or Voges et al. Development 2017; 144(6):1118-1127). In a preferred embodiment, the engineered cardiac tissue exhibits contractile activity of less than about 10 mN/mm2.

In one embodiment the hydrogel is comprised of fibrin, collagen I, or Matrigel, or a combination of any thereof. In a preferred embodiment the hydrogel is a fibrin hydrogel. In a preferred embodiment, fibrin is formed by mixing thrombin and fibrinogen solutions. Preferably, in the formation of the fibrin hydrogel, fibrinogen is present at a concentration of about 10 mg/mL-about 50 mg/mL. More preferably fibrinogen is present at a concentration of about 20 mg/mL.

In another embodiment, the hydrogel may be functionalised with one or more bioactive agents. For example, the bioactive agents (e.g. small molecules, polypeptides including cytokines and chemokines, differentiation factors, signalling pathway inhibitors etc.) may, for example, facilitate viability of the cells in the resultant engineered tissue and the further development or differentiation of cells. In one embodiment one or more bioactive agents may be agents selected from the group consisting of: anti-proliferative agents, immunosuppressants, pro-angiogenic compounds, antibodies or fragments or portions thereof, antibiotics or antimicrobial compounds, antigens or epitopes, aptamers, biopolymers, carbohydrates, cell attachment mediators (such as RGD), cytokines, cytotoxic agents, drugs, enzymes, growth factors or recombinant growth factors and fragments and variants thereof, hormone antagonists, hormones, immunological agents, lipids, metals, nanoparticles, nucleic acid analogs, nucleic acids (e.g., DNA, RNA, siRNA, RNAi, and microRNA agents), nucleotides, nutraceutical agents, oligonucleotides, peptide nucleic acids (PNA), peptides, prodrugs, prophylactic agents, proteins, small molecules, therapeutic agents, or any combinations thereof.

In one embodiment, the cell suspension is mixed with the hydrogel in step i) to provide a cell concentration of about 5×10⁶, about 10×10⁶, about 15×10⁶, about 20×10⁶, about 25×10⁶, about 30×10⁶, about 35×10⁶, about 40×10⁶, about 45×10⁶, about 50×10⁶, about 55×10⁶, about 60×10⁶, about 65×10⁶, about 70×10⁶, about 75×10⁶, about 80×10⁶, about 85×10⁶, about 90×10⁶, about 95×10⁶, or about 100×10⁶ cells/mL. More preferably, the cell suspension is mixed with the hydrogel in step i) to provide a cell concentration of about 5×10⁶ to about 50×10⁶ cells/mL. Even more preferably, the cell suspension is mixed with the hydrogel in step i) to provide a cell concentration of about 10×10⁶, about 15×10⁶, about 20×10⁶, about 25×10⁶, about 30×10⁶ or about 35×10⁶ cells/mL.

In one embodiment, the engineered human cardiac tissue has about 1×10⁶, 2×10⁶, 3×10⁶, 4×10⁶, 5×10⁶, about 10×10⁶, about 15×10⁶, about 20×10⁶, about 25×10⁶, about 30×10⁶, about 35×10⁶, about 40×10⁶, about 45×10⁶, about 50×10⁶, about 55×10⁶, about 60×10⁶, about 65×10⁶, about 70×10⁶, about 75×10⁶, about 80×10⁶, about 85×10⁶, about 90×10⁶, about 95×10⁶, about 100×10⁶, about 110×10⁶, about 120×10⁶, about 130×10⁶, about 140×10⁶, or about 150×10⁶ cells at the time the hydrogel is solidified. With reference to the method of the first aspect of the invention, it is the cell density of the patch at the end of step (ii) and before the hydrogel is cultured in serum free medium as defined in step (iii). More preferably, the engineered human cardiac tissue has about 1×10⁶ to about 100×10⁶ cells. Even more preferably, the engineered human cardiac tissue has about 20×10⁶ cells to about 150×10⁶ cells.

In one embodiment, the engineered human cardiac tissue has a cell density of about 1×10⁶×10⁶, about 2×10⁶, about 3×10⁶, about 4×10⁶, about 5×10⁶, about 10×10⁶, about 15×10⁶, about 20×10⁶, about 25×10⁶, about 30×10⁶, about 35×10⁶, about 40×10⁶, about 45×10⁶, about 50×10⁶, about 55×10⁶, about 60×10⁶, about 65×10⁶, about 70×10⁶, about 75×10⁶, about 80×10⁶, about 85×10⁶, about 90×10⁶, about 95×10⁶, or about 100×10⁶ cells/cm² at the time the hydrogel is solidified. With reference to the method of the first aspect of the invention, it is the cell density of the patch at the end of step (ii) and before the hydrogel is cultured in serum free medium as defined in step (iii). More preferably, the engineered human cardiac tissue has a cell density of about 1×10⁶ to about 50×10⁶ cells/cm². Even more preferably, the engineered human cardiac tissue has a cell density of about 1×10⁶ cells/cm² to about 10×10⁶ cells/cm².

The engineered human cardiac patch may be produced in any shape, size of configuration using an appropriate mold. In some embodiments, the tissue is shaped to reflect the site of application. In one embodiment the engineered cardiac tissue has a trapezoidal geometry.

The skilled person will be aware of various methods in the literature for directed differentiation of hPSCs to cardiac lineage to provide a mixed population of hPSC derived cardiomyocytes and non-cardiomyocyte cells. In one embodiment, prior to step i), hPSCs are induced to differentiate towards cardiac linage by the steps of:
a) culturing hPSCs in a basal medium comprising an effective amount of BMP4, Activin A, FGF, a GSK3-inhibitor for a period sufficient to induce mesoderm differentiation of the hPSCs;
b) culturing the cells obtained in step a) in a basal medium comprising an effective amount of an inhibitor of the Wnt-signaling pathway for a period sufficient to induce cardiac differentiation of the mesoderm;
c) culturing the cells obtained in step b) in a basal medium for a period sufficient to provide a mixed population of hPSC derived cardiomyocytes and non-cardiomyocyte cells.

In one aspect, the present invention provides a method for producing a population of human pluripotent stem cell (hPSC) culture-derived cardiomyocytes, stromal cells/fibroblasts and endothelial cells, the method comprising:
a) culturing hPSCs in a basal medium comprising an effective amount of BMP4, Activin A, FGF, a GSK3-inhibitor for a period sufficient to induce mesoderm differentiation of the hPSCs;
b) culturing the cells obtained in step a) in a basal medium comprising an effective amount of an inhibitor of the Wnt-signaling pathway for a period sufficient to induce cardiac differentiation of the mesoderm;
c) culturing the cells obtained in step b) in a basal medium for a period sufficient to provide a mixed population of hPSC derived cardiomyocytes and non-cardiomyocyte cells; and
d) culturing the cells obtained in step c) in a basal medium comprising an effective amount of an FGFR1 agonist and a PDGFR agonist for a period sufficient to produce a population of human pluripotent stem cell (hPSC) culture-derived cardiomyocytes, stromal cells/fibroblasts and endothelial cells.

In a preferred embodiment, prior to step i), hPSCs are induced to differentiate towards cardiac linage by the steps of:
a) culturing hPSCs in a basal medium comprising an effective amount of BMP4, Activin A, FGF, a GSK3-inhibitor, a serum-free supplement comprising albumin and transferrin minus insulin for about 72 h wherein the medium is replaced with fresh medium daily;
b) culturing the cells obtained in step a) in a basal medium comprising an effective amount of an inhibitor of the Wnt-signaling pathway and a serum-free supplement as in a) for about 72 h wherein the medium is replaced with fresh medium daily;
c) culturing the cells obtained in step b) in a basal medium comprising an effective amount of an inhibitor of the Wnt-signaling pathway and a serum-free supplement comprising albumin, and transferrin and including insulin, for a period of about 7 days wherein the medium is replaced with fresh medium after on day 2 and day 4 of said period of about 7 days;
d) culturing the cells obtained in step c) in a basal medium comprising albumin, transferrin and insulin for a period of about 72 h; wherein the basal medium used in each of steps a)-d) has a calcium concentration of less than about 1.2 mM;
   to thereby provide a cell suspension comprising a population of human pluripotent stem cell (hPSC) culture-derived cardiomyocytes and non-cardiomyocyte cells.

In one embodiment, in step a) the hPSCs are culture in a basal medium comprises 1-20 ng/ml BMP4, preferably 2-15 ng/ml, more preferably 2.5-10 ng/ml, more preferably 3-8 ng/ml, most preferably 4-6 ng/ml, and even most preferably about 5 ng/ml; 0.1-10 ng/ml FGF2, preferably 1-9 ng/ml, more preferably 2-8 ng/ml, even more preferably 3-7 ng/ml, most preferably 4-6 ng/ml, and even most preferably about 5 ng/ml; 1-20 ng/ml Activin A, preferably 2.5-18 ng/ml, more preferably 5-16 ng/ml, even more preferably 7.5-14 ng/ml, still more preferably 8-12 ng/ml, most preferably 8.5-10 ng/ml, and even most preferably about 9 ng/ml.

The GSK3-inhibitor in the basal medium of step (a) can be selected, for example, from the group consisting of CHIR99021, CHIR98014, SB216763, TWS119, Tideglusib, SB415286, and LY2090314. However, any GSK3-inhibitor suitable in the method of the invention can be applied. In a preferred embodiment, the GSK3-inhibitor in the basal medium of step (a) is CHIR99021.

It will be understood by the skilled person that the concentration of an effective amount of a GSK3-inhibitor varies with the availability and inhibition constant of the inhibitor in question. In the context of the present invention, the term “effective amount” as used herein in the context of a GSK3-inhibitor is intended to mean an enzyme inactivating concentration. For example, in case of CHIR99021, the basal medium in step (a) comprises 0.1-10 μM CHIR99021, preferably 0.2-9 μM, more preferably 0.3-8 μM, even more preferably 0.4-7 μM, still more preferably 0.5-6 μM, more preferably 0.6-5 μM, more preferably 0.7-4 μM, more preferably 0.8-3 μM, most preferably 0.9-2 μM, and even most preferably about 1 μM CHIR99021.

In one embodiment, the serum free supplement is a B27® supplement or B27® supplement minus insulin. In a preferred embodiment, the B27® supplement or B27® supplement minus insulin is applied in an amount of 0.1-10 % B27® or B27® minus insulin, preferably 0.5-8 %, more preferably 1-6 %, even more preferably 1.5-4%, and most preferably about 4% B27® or B27® minus insulin.

In one embodiment the Wnt pathway inhibitor is selected from the group consisting of C59 (4-(2-Methyl-4-pyridinyl)-N-[4-(3-pyridinyl)phenyl]benzeneacetamide), DKK1, IWP-2 (N-(6-Methyl-2-benzothiazolyl)-2-[(3,4,6,7-tetrahydro-4-oxo-3-phenylthieno[3,2-d]pyrimidin-2-yl)thio]-acetamide), IWP-4 (N-(6-Methyl-2-benzothiazolyl)-2-[(3,4,6,7-tetrahydro-3-(2-methoxyphenyl)-4-oxothieno[3,2-d]pyrimidin-2-yl)thio]-acetamide), Ant1.4Br, Ant1.4CI, Niclosamide, apicularen, bafilomycin, XAV939 (3,5,7,8-Tetrahydro-2-[4-(trifluoromethyl)phenyl]-4H-thiopyrano[4,3-d]pyrimidin-4-one), IWR-1 (4-(1,3,3a,4,7,7a-Hexahydro-1,3-dioxo-4,7-methano-2H-isoindol-2-yl)-N-8-quinolinyl-Benzamide), NSC668036 (N-[(1,1-Dimethylethoxy)carbonyl]-L-alanyl-(2S)-2-hydroxy-3-methylbutanoyl-L-Alanine-(1S)-1-carboxy-2-methylpropyl ester hydrate), 2,4-diamino-quinazoline, Quercetin, ICG-001 ((6S,9aS)-Hexahydro-6-[(4-hydroxyphenyl)methyl]-8-(1-naphthalenylmethyl)-4,7-dioxo-N-(phenylmethyl)-2H-pyrazino[1,2-a]pyrimidine-1(6H)-carboxamide), PKF115-584, BML-284 (2-Amino-4-[3,4-(methylenedioxy)benzylamino]-6-(3-methoxyphenyl)pyrimidine), FH-535, iCRT-14, JW-55, JW-67, antibodies to Wnts and Wnt receptors, and Wnt inhibitory nucleic acids. In a preferred embodiment the Wnt pathway inhibitor is IWP-4, IWR-1 or ICRT14.

Any pluripotent stem cell population, including a human embryonic stem cell population (hESC) or a human induced pluripotent stem cell population (iPSCs), can be used as the starting material to derive engineered human cardiac tissue using the methods described herein. In one embodiment, population of pluripotent progenitor cells is a human iPSC population. In one embodiment the iPSC cells grown in feeder-free conditions. In another embodiment, cells obtained from a subject can be subjected to methods to generate patient specific iPSCs which can then be differentiated using the methods described herein.

### Engineered Cardiac Tissues

The present invention provides engineered human cardiac tissue comprising at least about 30% hPSC-derived cardiomyocytes, about 35% or less hPSC-derived stromal cells/fibroblasts and up about 30% or less hPSC-derived endothelial cells, wherein the aforementioned cells are located throughout a hydrogel composition.

In another embodiment engineered human cardiac tissue comprising hPSC-derived cardiomyocytes, hPSC-derived stromal cells/fibroblasts and hPSC-derived endothelial cells, wherein the aforementioned cells are derived from a single differentiation process, wherein the aforementioned cells are located throughout a hydrogel composition.

The present invention provides engineered human cardiac tissue produced according to the methods as described hereinabove.

In some embodiments, the human engineered cardiac tissue comprises cardiomyocytes, stromal cells/fibroblasts, endothelial cells and vascular smooth muscle cells. In a preferred embodiment, the engineered cardiac tissue comprises at least about 30 % cardiomyocytes; about 35% or less stromal cells/fibroblasts; and about 30% or less endothelial cells. In one embodiment, the engineered cardiac tissue comprises about 30% to about 70% cardiomyocytes; about 10% to about 35% stromal cells/fibroblasts; and about 2% to about 30% endothelial cells. In another embodiment, the engineered cardiac tissue comprises about 30% to about 65% cardiomyocytes; about 10% to about 35% stromal cells/fibroblasts; and about 2% to about 30% endothelial cells. In another embodiment, the engineered cardiac tissue comprises about 30% to about 60% cardiomyocytes; about 10% to about 35% stromal cells/fibroblasts; and about 2% to about 30% endothelial cells. In another embodiment, the engineered cardiac tissue comprises about 30% to about 55% cardiomyocytes; about 10% to about 35% stromal cells/fibroblasts; and about 2% to about 30% endothelial cells. In another embodiment, the engineered cardiac tissue comprises about 30% to about 70% cardiomyocytes; about 15% to about 35% stromal cells/fibroblasts; and about 2% to about 30% endothelial cells. In another embodiment, the engineered cardiac tissue comprises about 30% to about 70% cardiomyocytes; about 20% to about 35% stromal cells/fibroblasts; and about 2% to about 30% endothelial cells. In another embodiment, the engineered cardiac tissue comprises about 30% to about 70% cardiomyocytes; about 10% to about 35% stromal cells/fibroblasts; and about 5% to about 30% endothelial cells. In another embodiment, the engineered cardiac tissue comprises about 30% to about 70% cardiomyocytes; about 10% to about 35% stromal cells/fibroblasts; and about 15% to about 30% endothelial cells. In a more preferred embodiment, the engineered cardiac tissue comprises about 30% to about 50% cardiomyocytes; about 20% to about 35% stromal cells/fibroblasts; and about 15% to about 30% endothelial cells. In a more preferred embodiment, the engineered cardiac tissue comprises about 30% to about 55% cardiomyocytes; about 20% to about 35% stromal cells/fibroblasts; and about 15% to about 30% endothelial cells. In some embodiments, the cardiac tissue further comprises up to about 15% vascular smooth muscle cells. In some embodiments, the cardiac tissue further comprises up to about 10% cardiac progenitor cells. In a more preferred embodiment, the engineered cardiac tissue comprises about 30% to about 70% cardiomyocytes; about 10% to about 35% stromal cells/fibroblasts; about 2% to about 30% endothelial cells; up to about 15% vascular smooth muscle cells; and up to about 10 % cardiac progenitor cells. In a more preferred embodiment, the engineered cardiac tissue comprises about 30% to about 60% cardiomyocytes; about 15% to about 35% stromal cells/fibroblasts; about 5% to about 30% endothelial cells; up to about 15% vascular smooth muscle cells; and up to about 10 % cardiac progenitor cells. In a more preferred embodiment, the engineered cardiac tissue comprises about 30% to about 55% cardiomyocytes; about 20% to about 35% stromal cells/fibroblasts; about 15% to about 30% endothelial cells; up to about 15% vascular smooth muscle cells; and up to about 10 % cardiac progenitor cells. In a more preferred embodiment, the engineered cardiac tissue comprises about 30% to about 55% cardiomyocytes; about 20% to about 35% stromal cells/fibroblasts; about 5% to about 30% endothelial cells; about 0.5% to about 15% vascular smooth muscle cells; and about 0.5% to about 10 % cardiac progenitor cells. In a more preferred embodiment, the engineered cardiac tissue comprises about 30% to about 55 % cardiomyocytes; about 20% to about 35% stromal cells/fibroblasts; about 15% to about 30% endothelial cells; about 0.5% to about 15% vascular smooth muscle cells; and about 0.5% to about 10 % cardiac progenitor cells. In some embodiments, the engineered cardiac tissue comprises about 30% to about 60% cardiomyocytes, about 20% to about 35% stromal cells/fibroblasts; and about 15% to about 30% endothelial cells. In some embodiments, the engineered cardiac tissue comprises about 30% to about 45% cardiomyocytes, about 30% to about 35% stromal cells/fibroblasts; and about 20% to about 24% endothelial cells. In some embodiments, the engineered cardiac tissue comprises about 35% to about 55% cardiomyocytes, about 20% to about 35% stromal cells/fibroblasts; and about 15% to about 30% endothelial cells. In some embodiments, the engineered cardiac tissue comprises about 35% to about 60% cardiomyocytes, about 20% to about 35% stromal cells/fibroblasts; and about 15% to about 30% endothelial cells. In some embodiments, the engineered cardiac tissue comprises about 35% to about 55% cardiomyocytes, about 40% to about 60% stromal cells/fibroblasts; and about 15% to about 30% endothelial cells. In some embodiments, the engineered cardiac tissue comprises about 30% to about 45% cardiomyocytes, about 30% to about 35% stromal cells/fibroblasts; and about 26% to about 30% endothelial cells. In some embodiments, the engineered cardiac tissue comprises about 30% to about 45% cardiomyocytes, about 30% to about 35% stromal cells/fibroblasts; and about 20% endothelial cells. In some embodiments, the engineered cardiac tissue comprises about 30% to about 45% cardiomyocytes, about 30% to about 35% stromal cells/fibroblasts; and about 30% endothelial cells.

In some embodiments, the engineered cardiac tissue comprises about 1% to about 10% proliferative cardiomyocytes. In some embodiments, the engineered cardiac tissue comprises about 2% to about 5% proliferative cardiomyocytes. In a further embodiment, at least about 3% to about 10% of the cardiomyocytes of the engineered cardiac tissue are proliferative as determined by the expression of one or more markers associated with cell proliferation. In a further embodiment, about 3%, about 4%, about 5%, about 6%, about 7%, about 8%, about 9%, or about 10% of the cardiomyocytes of the engineered cardiac tissue are proliferative as determined by the expression of one or more markers associated with cell proliferation. In one embodiment, the proliferative cardiomyocytes express one or more markers selected from the group consisting of ANLN, TOP2A, CDK1, and E2F1.

Another characteristic of the cardiomyocytes present in the engineered cardiac tissues is that they are MMP1⁺. In a preferred embodiment therefore, the cardiomyocytes in the engineered cardiac tissue are MMP1⁺.

Another characteristic of the fibroblasts present in the engineered cardiac tissues is that they are MMP1⁺, EMP1⁺, FOXD1⁺, RAB27B⁺, NR2F1⁺, F2RL1⁺, SPP1⁺, TMEM158⁺, PTHLH⁺, PHLDA2⁺, MALL⁺, MYCT1⁺, DUSP4⁺, PLAU+, TMEM156⁺, CD274⁺, MMP10⁺, ARRDC4⁺, RFX8⁺, MLPH⁺, THBD⁺, HHEX⁺, VGF⁺, OTULINL⁺, IL33⁺, CA12⁺, C6orf141⁺, MFSD2A⁺ and/or CARD10⁺. In a preferred embodiment therefore, the fibroblasts in the engineered cardiac tissue are positive for one or more of these aforementioned markers.

In one embodiment, the cardiac tissues described herein display log2 relative gene expression >2 for PKM (normalized to TUBA1A). Another characteristic of the cardiac tissues is that after 24 h in SF culture medium, the SF culture medium has a concentration of lactate of at least 0.5 mM or at least 1 mM. In another embodiment, after culture of the cardiac tissues generated using the methods described herein for 24 h in SF culture medium, the culture medium has a concentration of lactate of the 10-20 nmol/million cells/h.

In one embodiment, the engineered cardiac tissues display tolerance to hypoxia. In one embodiment, the cardiac tissues described herein display log2 relative gene expression of <−4 for ALDH1, AGMO, GPX3, CYP1B1 and PLIN5 (normalized to TUBA1A). Another characteristic of the engineered cardiac tissues is that after 20 h of culture under hypoxic conditions, cell culture medium from such culture has a lactate/pyruvate ratio of at least 1:1.

As demonstrated in the examples below, it has been proven advantageous that the engineered cardiac tissues generated by methods of the present invention display low contractility. In a preferred embodiment, the engineered cardiac tissue exhibits contractile activity of less than about 10 mN/mm2.

In one embodiment the hydrogel composition is comprised of fibrin, collagen I, or Matrigel, or a combination of any thereof. In a preferred embodiment the hydrogel is a fibrin hydrogel. In another embodiment, the hydrogel may be functionalised with one or more bioactive agents. For example, the bioactive agents (e.g. small molecules, polypeptides including cytokines and chemokines, differentiation factors, signalling pathway inhibitors etc.) may, for example, facilitate viability of the cells in the resultant engineered tissue and the further development or differentiation of cells. In one embodiment one or more bioactive agents may be agents selected from the group consisting of: anti-proliferative agents, immunosuppressants, pro-angiogenic compounds, antibodies or fragments or portions thereof, antibiotics or antimicrobial compounds, antigens or epitopes, aptamers, biopolymers, carbohydrates, cell attachment mediators (such as RGD), cytokines, cytotoxic agents, drugs, enzymes, growth factors or recombinant growth factors and fragments and variants thereof, hormone antagonists, hormones, immunological agents, lipids, metals, nanoparticles, nucleic acid analogs, nucleic acids (e.g., DNA, RNA, siRNA, RNAi, and microRNA agents), nucleotides, nutraceutical agents, oligonucleotides, peptide nucleic acids (PNA), peptides, prodrugs, prophylactic agents, proteins, small molecules, therapeutic agents, or any combinations thereof.

In one embodiment, the engineered human cardiac tissue has about 1×10⁶, 2×10⁶, 3×10⁶, 4×10⁶, 5×10⁶, about 10×10⁶, about 15×10⁶, about 20×10⁶, about 25×10⁶, about 30×10⁶, about 35×10⁶, about 40×10⁶, about 45×10⁶, about 50×10⁶, about 55×10⁶, about 60×10⁶, about 65×10⁶, about 70×10⁶, about 75×10⁶, about 80×10⁶, about 85×10⁶, about 90×10⁶, about 95×10⁶, about 100×10⁶, about 110×10⁶, about 120×10⁶, about 130×10⁶, about 140×10⁶, or about 150×10⁶ cells. More preferably, the engineered human cardiac tissue has about 1×10⁶ to about 100×10⁶ cells. Even more preferably, the engineered human cardiac tissue has about 20×10⁶ cells to about 150×10⁶ cells.

In one embodiment, the engineered human cardiac tissue has a cell density of about 1×10⁶, about 2×10⁶, about 3×10⁶, about 4×10⁶, about 5×10⁶, about 10×10⁶, about 15×10⁶, about 20×10⁶, about 25×10⁶, about 30×10⁶, about 35×10⁶, about 40×10⁶, about 45×10⁶, about 50×10⁶, about 55×10⁶, about 60×10⁶, about 65×10⁶, about 70×10⁶, about 75×10⁶, about 80×10⁶, about 85×10⁶, about 90×10⁶, about 95×10⁶, or about 100×10⁶ cells/cm². More preferably, the engineered human cardiac tissue has a cell density of about 1×10⁶ to about 50×10⁶ cells/cm². Even more preferably, the engineered human cardiac tissue has a cell density of about 1×10⁶ cells/cm² to about 10×10⁶ cells/cm².

The engineered human cardiac patch may be produced in any shape, size of configuration using an appropriate mold. In some embodiments, the tissue is shaped to reflect the site of application. In one embodiment the engineered cardiac tissue has a trapezoidal geometry.

The engineered human cardiac tissue may be derived from a human embryonic stem cell population (hESC) or a human induced pluripotent stem cell population (iPSCs), can be used as the starting material. In one embodiment, the engineered human cardiac tissue is derived from a human iPSC population. In one embodiment the iPSC cells grown in feeder-free conditions. In another embodiment, engineered human cardiac tissue is derived from cells obtained from a patient and subjected to methods to generate patient specific iPSCs which can then be differentiated using the methods described herein to generate engineered human cardiac tissue.

### Methods of Treatment

To engineer human cardiac tissue for the purposes of transplantation into heart disease and heart failure patients, there is a need to provide a cardiac tissue patch that has a biocompatible structure that is sufficiently durable and amenable for transplantation and can withstand transplantation into what is a hypoxic environment. There is also a need to provide an engineered cardiac tissue that possesses an appropriate variety of cardiomyocyte and non-cardiomyocyte cell types, in particular endothelial cells, such that transplanted cardiac tissue patches can be vascularised, remain viable and ultimately functionally integrate into the recipient heart. Accordingly, a better tissue for transplantation is required.

As described herein, the present inventors have also surprisingly identified that through the specific culture conditions, hPSC-derived cardiac tissue patches are generated comprising cardiomyocytes, stromal cells/fibroblasts and increased numbers of endothelial cells that are of sufficient durability for transplantation and have increased tolerance to hypoxia. Without wishing to be bound by any particular theory, in producing the engineered cardiac tissue according to the methods described herein, the differentiation of progenitor cells towards an endothelial cell fate as well as pericytes which serve to support the ECs is promoted, and an engineered tissue having predominantly glycolytic metabolism with reduced oxidation, including reduced fatty acid oxidation, and low contractility is provided. This results in an engineered tissue that is more resistant to hypoxia and may indicate that such engineered cardiac tissue is more suitable for therapeutic applications such as transplantation.

The present invention therefore provides engineered human cardiac tissue as described and defined herein for use as an implant in the treatment of diseased or damaged cardiac tissue in a subject in need thereof.

The present invention also provides a method for treating diseased or damaged cardiac tissue in a subject in need thereof comprising implanting the engineered human cardiac tissue as described and defined herein in said subject.

The present invention also provides use of engineered human cardiac tissue as described and defined herein in the manufacture of a medicament for the treatment of diseased or damaged cardiac tissue in a subject in need thereof.

In one embodiment, the subject is suffering from cardiomyopathy or a cardiac tissue injury. In another embodiment, the cardiomyopathy or cardiac tissue injury is due to acute or chronic stress, atheromatous disorders of blood vessels, ischemia, myocardial infarction, inflammatory disease, heart valve disease, or myocarditis. In a further embodiment, the subject is suffering from a congenital heart disease. In yet a further embodiment, the congenital heart disease is selected from a group consisting of single ventricle disorders including hypoplastic left heart syndrome, tetralogy of fallot, truncus arteriosus, pulmonary atresia, ventricular septal defects, atrial septal defects, and endocardial cushion defect.

### Screening Methods

The present invention also provides for the use of the engineered human cardiac tissue according to the invention in an in vitro-model for drug toxicity screening or an in vitro method for testing of cardiac function modulation by candidate pharmacological agents. In one embodiment, the invention provides a method for analysing the biological effect of at least one test compound or bioactive agent on cardiac cells, comprising contacting the engineered human cardiac tissue with the test compound or bioactive agent, incubating the tissue in the presence of the test compound or bioactive agent, and analysing the biological effect.

### Kits

Also provided herein are kits comprising one or more of a cell or tissue generated according to a method described herein, a product or composition comprising a cell or tissue generated, optionally comprising a further therapeutic agent, or optionally wherein the cell comprises a reporter system or other modification, according to a method described herein, a combination of at least two selected from an agonist, inhibitor, media, apparatus or other component that can be used in a method described herein and instructions for use, for example instructions on how to generate the cells, perform an assay or administer the cell, tissue, composition, or product, and a vial or other container for housing one of these aforementioned cells, tissues, compositions, products, agonists, inhibitors, medias etc.

### List of Numbered Embodiments

1. A method for producing engineered human cardiac tissue comprising endothelial cells, the method comprising culturing a cell suspension comprising a population of human pluripotent stem cell (hPSC) culture-derived cardiomyocytes and non-cardiomyocyte cells in the presence of a FGFR1 agonist and a PDGFR agonist for a time and under conditions sufficient to produce engineered human cardiac tissue comprising endothelial cells.
2. A method for producing engineered human cardiac tissue, the method comprising the steps of:
   i) mixing a cell suspension comprising a population of human pluripotent stem cell (hPSC) culture-derived cardiomyocytes and non-cardiomyocyte cells with a flowable hydrogel composition, wherein said cell suspension is in a serum-free medium;
   ii) loading the flowable hydrogel composition comprising said cell suspension into a mold and incubating the hydrogel for a time and under conditions sufficient to solidify the hydrogel; and
   iii) culturing the hydrogel from step ii) in serum free medium;
      wherein the population of human pluripotent stem cell (hPSC) culture-derived cardiomyocytes and non-cardiomyocyte cells is contacted with an FGFR1 agonist and a PDGFR agonist to thereby provide engineered human cardiac tissue, wherein said tissue comprises cardiomyocytes, stromal cells/fibroblasts and endothelial cells.
3. The method of embodiment 2, wherein prior to mixing in step i), said FGFR1 agonist and/or said PDGFR agonist are added to the serum free medium or the flowable hydrogel composition.
4. The method of embodiment 2, wherein after mixing in step i), said FGFR1 agonist and/or said PDGFR agonist are added to the flowable hydrogel composition comprising said cell suspension.
5. The method of any one of embodiments 2-4, wherein prior to mixing in step i), the population of human pluripotent stem cell (hPSC) culture-derived cardiomyocytes and non-cardiomyocyte cells are cultured in a serum free medium comprising a FGFR1 agonist and a PDGFR agonist.
6. The method of any one of embodiments 2-5, wherein in step iii) the hydrogel of step ii) is cultured in a serum free medium comprising the FGFR1 agonist and the PDGFR agonist.
7. The method of any one of the preceding embodiments, wherein said cell suspension comprises about 70% cardiomyocytes and about 30% to about 50% non-cardiomyocyte cells.
8. The method of any one of the preceding embodiments, wherein said cell suspension comprises about 50 to about 75% cardiomyocytes and about 25 to about 50% non-cardiomyocyte cells.
9. The method of any one of the preceding embodiments, wherein said cell suspension is derived from a single differentiation process.
10. The method of any one of the preceding embodiments, wherein the engineered human cardiac tissue further comprises vascular smooth muscle cells and cardiac progenitor cells.
11. The method of any one of the preceding embodiments, wherein the engineered human cardiac tissue comprises:
   at least about 30 % cardiomyocytes;
   about 35% or less stromal cells/fibroblasts; and
   about 30% or less endothelial cells.
12. The method of any one of the preceding embodiments, wherein the engineered human cardiac tissue comprises:
   about 30 to about 70% cardiomyocytes;
   about 10 to about 35% stromal cells/fibroblasts;
   about 5 to about 30% endothelial cells;
   up to about 15% vascular smooth muscle cells; and
   up to about 10 % cardiac progenitor cells.
13. The method of any one of the preceding embodiments, wherein the FGFR1 agonist is selected from FGF1, FGF2, FGF3, FGF4, FGF5, FGF6, FGF7, FGF8, FGF9, FGF10, FGF16, FGF17, FGF18, FGF19, FGF20, FGF21, FGF22, and FGF23.
14. The method of embodiment 13, wherein the FGFR1 agonist is FGF2.
15. The method of any one of the preceding embodiments, wherein the PDGFR agonist is selected from PDGF-AA, PDGF-BB, PDGF-AB, PDGF-CC or PDGF-DD.
16. The method of embodiment 15, wherein the PDGFR agonist is a PDGFRβ agonist.
17. The method of embodiment 16, wherein the PDGFR agonist is PDGF-BB.
18. The method of any one of the preceding embodiments, wherein the serum free medium comprises about 0.5-100 ng/ml human PDGF-BB and about 0.5-100 ng/ml human FGF2.
19. The method of any one of the preceding embodiments wherein the serum free medium comprises about 10 ng/ml human PDGF-BB and about 10 ng/ml human FGF2.
20. The method of any one of the preceding embodiments, wherein said serum free medium comprises a basal medium comprising albumin and transferrin.
21. The method of embodiment 20, wherein said serum free medium comprises B27 supplement.
22. The method of embodiment 20 or 21, wherein said basal medium is a low calcium medium having a calcium concentration of less than about 1.2 mM.
23. The method of any one of embodiments 2-22, further comprising replacing the medium in step iii) for fresh cell culture medium at least every 2 days.
24. The method of any one of embodiments 2-23, wherein the culturing in step iii) is carried out for at least 5 days.
25. The method of any one of the preceding embodiments, wherein a population of about 3% to about 10% of the cardiomyocytes in said engineered human cardiac tissue are proliferative.
26. The method of any one of the preceding embodiments, wherein said engineered human cardiac tissue displays glycolytic metabolism.
27. The method of embodiment 26, wherein cell culture medium obtained following 24 h culture with said engineered human cardiac tissue has a concentration of lactate of 1 mM.
28. The method of any one of the preceding embodiments, wherein said engineered human cardiac tissue displays log2 relative gene expression of >2 for PKM, when normalized to TUBA1A expression.
29. The method of any one of the preceding embodiments, wherein said engineered human cardiac tissue has tolerance to hypoxia.
30. The method of any one of the preceding embodiments, wherein said engineered human cardiac tissue yields a lactate/pyruvate ratio of at least 1:1 in cell culture medium obtained following 20 h of culture under hypoxic conditions.
31. The method of any one of the preceding embodiments, wherein said engineered human cardiac tissue displays downregulation of ALDH1, AGMO, DPYD, GPX3, CYP1B1 and PLIN5.
32. The method of any one of the preceding embodiments, wherein the cardiomyocytes are MMP1⁺.
33. The method of any one of the preceding embodiments, wherein the fibroblasts are MMP1⁺, EMP1⁺, FOXD1⁺, RAB27B⁺, NR2F1⁺, F2RL1⁺, SPP1⁺, TMEM158⁺, PTHLH⁺, PHLDA2⁺, MALL⁺, MYCT1⁺, DUSP4⁺, PLAU⁺, TMEM156⁺, CD274⁺, MMP10⁺, ARRDC4⁺, RFX8⁺, MLPH⁺, THBD⁺, HHEX⁺, VGF⁺, OTULINL⁺, IL33⁺, CA12⁺, C6orf141⁺, MFSD2A⁺ and/or CARD10⁺.
34. The method of any one of the preceding embodiments, wherein said engineered human cardiac tissue exhibits contractile activity of less than 10 mN/mm2.
35. The method of any one of embodiments 2-34, wherein the hydrogel composition comprises a fibrin hydrogel.
36. The method of embodiment 35, wherein fibrin is formed by mixing thrombin and fibrinogen solutions.
37. The method of embodiment 36, wherein fibrinogen is present at a concentration of 10 mg/mL-50 mg/mL.
38. The method of embodiment 36, wherein fibrinogen is present at a concentration of 20 mg/mL.
39. The method of any one of embodiments 2-38, wherein the cell suspension is mixed with the hydrogel in step i) to provide a cell concentration of about 5×10⁶ to about 100×10⁶ cells/mL.
40. The method of embodiment 39, wherein the cell suspension is mixed with the hydrogel in step i) to provide a cell concentration of about 20×10⁶ to about 50×10⁶ cells/mL.
41. The method of embodiment 40, wherein the cell suspension is mixed with the hydrogel in step i) to provide a cell concentration of about 35×10⁶ cells/mL.
42. The method of any one of the preceding embodiments, hPSCs are induced to differentiate towards cardiac linage by the steps of:
   a) culturing hPSCs in a basal medium comprising an effective amount of BMP4, Activin A, FGF, a GSK3-inhibitor, a serum-free supplement comprising albumin and transferrin minus insulin for about 72 h wherein the medium is replaced with fresh medium daily;
   b) culturing the cells obtained in step a) in a basal medium comprising an effective amount of an inhibitor of the Wnt-signallng pathway and a serum-free supplement as in a) for about 72 h wherein the medium is replaced with fresh medium daily,
   c) culturing the cells obtained in step b) in a basal medium comprising an effective amount of an inhibitor of the Wnt-signallng pathway and a serum-free supplement comprising albumin and transferrin including insulin for a period of about 7 days wherein the medium is replaced with fresh medium after on day 2 and day 4 of said period of about 7 days;
   d) culturing the cells obtained in step c) in a basal medium comprising a serum-free supplement comprising albumin, transferrin and insulin for a period of about 72 h;
      wherein the basal medium used in each of steps a)-d) has a calcium concentration of less than about 1.2 mM;
      to thereby provide a cell suspension comprising a population of human pluripotent stem cell (hPSC) culture-derived cardiomyocytes and non-cardiomyocyte cells.
43. The method of embodiment 42, wherein:
   said GSK3-inhibitor is CHIR99021; and/or
   said inhibitor of the Wnt-signallng pathway is IWP-4; and/or
   said basal medium is RPMI or DMEM.
44. The method of any one of the preceding embodiments, wherein the engineered human cardiac tissue is in the form of a tissue patch.
45. The method of embodiment 44, wherein the patch is employed as an implant in a subject.
46. The method of embodiment 45, wherein the subject is suffering from cardiomyopathy or a cardiac tissue injury.
47. The method of embodiment 46, wherein the cardiomyopathy or cardiac tissue injury is due to acute or chronic stress, atheromatous disorders of blood vessels, ischemia, myocardial infarction, inflammatory disease, heart valve disease, or myocarditis.
48. The method of embodiment 45, wherein the subject is suffering from a congenital heart disease.
49. The method of embodiment 48, wherein the congenital heart disease is selected from a group consisting of single ventricle disorders including hypoplastic left heart syndrome, tetralogy of fallot, truncus arteriosus, pulmonary atresia, ventricular septal defects, atrial septal defects, and endocardial cushion defect.
50. The method of any one of the preceding embodiments, wherein said hPSCs are iPSCs.
51. The method of embodiment 50, wherein said iPSCs are derived from a subject with heart disease and/or to be implanted with said engineered human cardiac tissue.
52. The method of any one of the preceding embodiments, wherein said hPSCs are embryonic stem cells.
53. Engineered human cardiac tissue comprising at least about 30% hPSC-derived cardiomyocytes, about 35% or less hPSC-derived stromal cells/fibroblasts and about 30% or less hPSC-derived endothelial cells, wherein the aforementioned cells are located throughout a hydrogel composition.
54. Engineered human cardiac tissue comprising hPSC-derived cardiomyocytes, hPSC-derived stromal cells/fibroblasts and hPSC-derived endothelial cells, wherein the aforementioned cells are derived from a single differentiation process, wherein the aforementioned cells are located throughout a hydrogel composition.
55. The engineered human cardiac tissue of embodiment 53 or 54, wherein the engineered human cardiac tissue further comprises vascular smooth muscle cells and cardiac progenitor cells.
56. The engineered human cardiac tissue of any one of embodiments 53 to 55, wherein the engineered human cardiac tissue comprises:
   about 30 to about 70 % cardiomyocytes;
   about 10 to about 35% stromal cells/fibroblasts; and
   about 5 to about 30% endothelial cells.
57. The engineered human cardiac tissue of any one of embodiments 53 to 56, wherein up to about 10% of the cardiomyocytes in said engineered human cardiac tissue are proliferative.
58. The engineered human cardiac tissue of any one of embodiments 53 to 57, wherein the engineered human cardiac tissue displays glycolytic metabolism.
59. The engineered human cardiac tissue of embodiment 53, wherein cell culture medium obtained following 24 h culture with said engineered human cardiac tissue has a concentration of lactate of 1 mM.
60. The engineered human cardiac tissue of any one of embodiments 53 to 59, wherein said engineered human cardiac tissue displays log2 relative gene expression of >2 of PKM when normalised to TUBA1A.
61. The engineered human cardiac tissue of any one of embodiments 53 to 60, wherein said engineered human cardiac tissue has tolerance to hypoxia.
62. The engineered human cardiac tissue of any one of embodiments 53 to 61, wherein said engineered human cardiac tissue yields a lactate/pyruvate ratio of at least 1:1 in cell culture medium obtained following 20 h of culture under hypoxic conditions.
63. The engineered human cardiac tissue of any one of embodiments 53 to 62, wherein said engineered human cardiac tissue displays log2 relative gene expression of <−4 of ALDH1, AGMO, GPX3, CYP1B1 and PLIN5 when normalised to TUBA1A expression.
64. The engineered human cardiac tissue of any one of embodiments 53 to 63, wherein the cardiomyocytes are MMP1⁺.
65. The engineered human cardiac tissue of any one of embodiments 53 to 64, wherein the fibroblasts are MMP1⁺, EMP1⁺, FOXD1⁺, RAB27B⁺, NR2F1⁺, F2RL1⁺, SPP1⁺, TMEM158⁺, PTHLH⁺, PHLDA2⁺, MALL⁺, MYCT1⁺, DUSP4⁺, PLAU⁺, TMEM156⁺, CD274⁺, MMP10⁺, ARRDC4⁺, RFX8⁺, MLPH⁺, THBD⁺, HHEX⁺, VGF⁺, OTULINL⁺, IL33⁺, CA12⁺, C6orf141⁺, MFSD2A⁺ and/or CARD10⁺.
66. The engineered human cardiac tissue of any one of embodiments 53 to 65 comprising:
   about 30 to about 70% cardiomyocytes;
   about 10 to about 35% stromal cells/fibroblasts;
   about 5 to about 30% endothelial cells;
   up to about 15% vascular smooth muscle cells; and
   up to about 10 % cardiac progenitor cells.
67. The engineered human cardiac tissue of any one of embodiments 53 to 66, wherein said engineered human cardiac tissue exhibits contractile activity of less than 10 mN/mm².
68. The engineered human cardiac tissue of any one of embodiments 53 to 67, wherein the hydrogel composition comprises fibrin formed by mixing thrombin and fibrinogen solutions.
69. The engineered human cardiac tissue of embodiment 68, wherein fibrinogen is present at a concentration of 10 mg/mL-50 mg/mL.
70. The engineered human cardiac tissue of embodiment 69, wherein fibrinogen is present at a concentration of 20 mg/mL.
71. The engineered human cardiac tissue of any one of embodiment 53 to 70, wherein the total number of cells in the tissue is about 20×10⁶ cells to 100×10⁶ cells.
72. The engineered human cardiac tissue of any one of embodiments 53 to 71, wherein the engineered human cardiac tissue is in the form of a tissue patch.
73. The engineered human cardiac tissue of any one of embodiments 53 to 72, wherein said hPSCs are iPSCs.
74. The engineered human cardiac tissue of any one of embodiments 53 to 73, wherein said hPSCs are embryonic stem cells.
75. Engineered human cardiac tissue produced according to the method of any one of embodiments 1 to 52.
76. A method for analysing the biological effect of at least one test compound or bioactive agent on cardiac cells, comprising contacting engineered human cardiac tissue of one of embodiments 53 to 75 with the test compound or bioactive agent, incubating the tissue in the presence of the test compound or bioactive agent, and analysing the biological effect.
77. Engineered human cardiac tissue of one of embodiments 53 to 75 for use as an implant in the treatment of diseased or damaged cardiac tissue in a subject in need thereof.
78. A method for treating diseased or damaged cardiac tissue in a subject in need thereof comprising implanting the engineered human cardiac tissue of one of embodiments 53 to 75 in said subject.
79. Use of engineered human cardiac tissue of one of embodiments 53 to 75 in the manufacture of a medicament for the treatment of diseased or damaged cardiac tissue in a subject in need thereof.
80. The engineered human cardiac tissue of embodiment 77, the method of embodiment 78 or the use of embodiment 79, wherein the subject is suffering from cardiomyopathy or a cardiac tissue injury.
81. The engineered human cardiac tissue, the method or the use of embodiment 80, wherein the cardiomyopathy or cardiac tissue injury is due to acute or chronic stress, atheromatous disorders of blood vessels, ischemia, myocardial infarction, inflammatory disease, heart valve disease, or myocarditis.
82. The engineered human cardiac tissue of embodiment 77, the method of embodiment 78 or the use of embodiment 79, wherein the subject is suffering from a congenital heart disease.
83. The engineered human cardiac tissue, the method or the use of embodiment 82, wherein the congenital heart disease is selected from a group consisting of single ventricle disorders including hypoplastic left heart syndrome, tetralogy of fallot, truncus arteriosus, pulmonary atresia, ventricular septal defects, atrial septal defects, and endocardial cushion defect.

1. A method for producing engineered human cardiac tissue comprising endothelial cells, the method comprising culturing a cell suspension comprising a population of human pluripotent stem cell (hPSC) culture-derived cardiomyocytes and non-cardiomyocyte cells in the presence of a FGFR1 agonist and a PDGFR agonist for a time and under conditions sufficient to produce engineered human cardiac tissue comprising endothelial cells.

2. A method for producing engineered human cardiac tissue, the method comprising the steps of:
i) mixing a cell suspension comprising a population of human pluripotent stem cell (hPSC) culture-derived cardiomyocytes and non-cardiomyocyte cells with a flowable hydrogel composition, wherein said cell suspension is in a serum-free medium;
ii) loading the flowable hydrogel composition comprising said cell suspension into a mold and incubating the hydrogel for a time and under conditions sufficient to solidify the hydrogel; and
iii) culturing the hydrogel from step ii) in serum free medium;
wherein the population of human pluripotent stem cell (hPSC) culture-derived cardiomyocytes and non-cardiomyocyte cells is contacted with an FGFR1 agonist and a PDGFR agonist to thereby provide engineered human cardiac tissue, wherein said tissue comprises cardiomyocytes, stromal cells/fibroblasts and endothelial cells.

3. The method of claim 2, wherein prior to mixing in step i), said FGFR1 agonist and/or said PDGFR agonist are added to the serum free medium or the flowable hydrogel composition.

4. The method of claim 2, wherein after mixing in step i), said FGFR1 agonist and/or said PDGFR agonist are added to the flowable hydrogel composition comprising said cell suspension.

5. The method of any one of claims 2-4, wherein prior to mixing in step i), the population of human pluripotent stem cell (hPSC) culture-derived cardiomyocytes and non-cardiomyocyte cells are cultured in a serum free medium comprising a FGFR1 agonist and a PDGFR agonist.

6. The method of any one of claims 2-5, wherein in step iii) the hydrogel of step ii) is cultured in a serum free medium comprising the FGFR1 agonist and the PDGFR agonist.

7. The method of any one of the preceding claims, wherein said cell suspension comprises about 70% cardiomyocytes and about 30% to about 50% non-cardiomyocyte cells.

8. The method of any one of the preceding claims, wherein said cell suspension comprises about 50 to about 75% cardiomyocytes and about 25 to about 50% non-cardiomyocyte cells.

9. The method of any one of the preceding claims, wherein said cell suspension is derived from a single differentiation process.

10. The method of any one of the preceding claims, wherein the engineered human cardiac tissue further comprises vascular smooth muscle cells and cardiac progenitor cells.

11. The method of any one of the preceding claims, wherein the engineered human cardiac tissue comprises:
at least about 30 % cardiomyocytes;
about 35% or less stromal cells/fibroblasts; and
about 30% or less endothelial cells.

12. The method of any one of the preceding claims, wherein the engineered human cardiac tissue comprises:
about 30 to about 70 % cardiomyocytes;
about 10 to about 35% stromal cells/fibroblasts;
about 5 to about 30% endothelial cells;
up to about 15% vascular smooth muscle cells; and
up to about 10 % cardiac progenitor cells.

13. The method of any one of the preceding claims, wherein the FGFR1 agonist is selected from FGF1, FGF2, FGF3, FGF4, FGF5, FGF6, FGF7, FGF8, FGF9, FGF10, FGF16, FGF17, FGF18, FGF19, FGF20, FGF21, FGF22, and FGF23.

14. The method of claim 13, wherein the FGFR1 agonist is FGF2.

15. The method of any one of the preceding claims, wherein the PDGFR agonist is selected from PDGF-AA, PDGF-BB, PDGF-AB, PDGF-CC or PDGF-DD.

16. The method of claim 15, wherein the PDGFR agonist is a PDGFRβ agonist.

17. The method of claim 16, wherein the PDGFR agonist is PDGF-BB.

18. The method of any one of the preceding claims, wherein the serum free medium comprises about 0.5-100 ng/ml human PDGF-BB and about 0.5-100 ng/ml human FGF2.

19. The method of any one of the preceding claims wherein the serum free medium comprises about 10 ng/ml human PDGF-BB and about 10 ng/ml human FGF2.

20. The method of any one of the preceding claims, wherein said serum free medium comprises a basal medium comprising albumin and transferrin.

21. The method of claim 20, wherein said serum free medium comprises B27 supplement.

22. The method of claim 20 or 21, wherein said basal medium is a low calcium medium having a calcium concentration of less than about 1.2 mM.

23. The method of any one of claims 2-22, further comprising replacing the medium in step iii) for fresh cell culture medium at least every 2 days.

24. The method of any one of claims 2-23, wherein the culturing in step iii) is carried out for at least 5 days.

25. The method of any one of the preceding claims, wherein a population of about 3% to about 10% of the cardiomyocytes in said engineered human cardiac tissue are proliferative.

26. The method of any one of the preceding claims, wherein said engineered human cardiac tissue displays glycolytic metabolism.

27. The method of claim 26, wherein cell culture medium obtained following 24 h culture with said engineered human cardiac tissue has a concentration of lactate of 1 mM.

28. The method of any one of the preceding claims, wherein said engineered human cardiac tissue displays log2 relative gene expression of >2 for PKM, when normalized to TUBA1A expression.

29. The method of any one of the preceding claims, wherein said engineered human cardiac tissue has tolerance to hypoxia.

30. The method of any one of the preceding claims, wherein said engineered human cardiac tissue yields a lactate/pyruvate ratio of at least 1:1 in cell culture medium obtained following 20 h of culture under hypoxic conditions.

31. The method of any one of the preceding claims, wherein said engineered human cardiac tissue displays downregulation of ALDH1, AGMO, DPYD, GPX3, CYP1B1 and PLIN5.

32. The method of any one of the preceding claims, wherein the cardiomyocytes are MMP1⁺.

33. The method of any one of the preceding claims, wherein the fibroblasts are MMP1⁺, EMP1⁺, FOXD1⁺, RAB27B⁺, NR2F1⁺, F2RL1⁺, SPP1⁺, TMEM158⁺, PTHLH⁺, PHLDA2⁺, MALL⁺, MYCT1⁺, DUSP4⁺, PLAU⁺, TMEM156⁺, CD274⁺, MMP10⁺, ARRDC4⁺, RFX8⁺, MLPH⁺, THBD⁺, HHEX⁺, VGF⁺, OTULINL⁺, IL33⁺, CA12⁺, C6orf141⁺, MFSD2A⁺ and/or CARD10⁺.

34. The method of any one of the preceding claims, wherein said engineered human cardiac tissue exhibits contractile activity of less than 10 mN/mm2.

35. The method of any one of claims 2-34, wherein the hydrogel composition comprises a fibrin hydrogel.

36. The method of claim 35, wherein fibrin is formed by mixing thrombin and fibrinogen solutions.

37. The method of claim 36, wherein fibrinogen is present at a concentration of 10 mg/mL-50 mg/mL.

38. The method of claim 36, wherein fibrinogen is present at a concentration of 20 mg/mL.

39. The method of any one of claims 2-38, wherein the cell suspension is mixed with the hydrogel in step i) to provide a cell concentration of about 5×10⁶ to about 100×10⁶ cells/mL.

40. The method of claim 39, wherein the cell suspension is mixed with the hydrogel in step i) to provide a cell concentration of about 20×10⁶ to about 50×10⁶ cells/mL.

41. The method of claim 40, wherein the cell suspension is mixed with the hydrogel in step i) to provide a cell concentration of about 35×10⁶ cells/mL.

42. The method of any one of the preceding claims, hPSCs are induced to differentiate towards cardiac linage by the steps of:
a) culturing hPSCs in a basal medium comprising an effective amount of BMP4, Activin A, FGF, a GSK3-inhibitor, a serum-free supplement comprising albumin and transferrin minus insulin for about 72 h wherein the medium is replaced with fresh medium daily;
b) culturing the cells obtained in step a) in a basal medium comprising an effective amount of an inhibitor of the Wnt-signaling pathway and a serum-free supplement as in a) for about 72 h wherein the medium is replaced with fresh medium daily,
c) culturing the cells obtained in step b) in a basal medium comprising an effective amount of an inhibitor of the Wnt-signaling pathway and a serum-free supplement comprising albumin and transferrin including insulin for a period of about 7 days wherein the medium is replaced with fresh medium after on day 2 and day 4 of said period of about 7 days;
d) culturing the cells obtained in step c) in a basal medium comprising a serum-free supplement comprising albumin, transferrin and insulin for a period of about 72 h;
wherein the basal medium used in each of steps a)-d) has a calcium concentration of less than about 1.2 mM;
to thereby provide a cell suspension comprising a population of human pluripotent stem cell (hPSC) culture-derived cardiomyocytes and non-cardiomyocyte cells.

43. The method of claim 42, wherein:
said GSK3-inhibitor is CHIR99021; and/or
said inhibitor of the Wnt-signallng pathway is IWP-4; and/or
said basal medium is RPMI or DMEM.

44. The method of any one of the preceding claims, wherein the engineered human cardiac tissue is in the form of a tissue patch.

45. The method of claim 44, wherein the patch is employed as an implant in a subject.

46. The method of claim 45, wherein the subject is suffering from cardiomyopathy or a cardiac tissue injury.

47. The method of claim 46, wherein the cardiomyopathy or cardiac tissue injury is due to acute or chronic stress, atheromatous disorders of blood vessels, ischemia, myocardial infarction, inflammatory disease, heart valve disease, or myocarditis.

48. The method of claim 45, wherein the subject is suffering from a congenital heart disease.

49. The method of claim 48, wherein the congenital heart disease is selected from a group consisting of single ventricle disorders including hypoplastic left heart syndrome, tetralogy of fallot, truncus arteriosus, pulmonary atresia, ventricular septal defects, atrial septal defects, and endocardial cushion defect.

50. The method of any one of the preceding claims, wherein said hPSCs are iPSCs.

51. The method of claim 50, wherein said iPSCs are derived from a subject with heart disease and/or to be implanted with said engineered human cardiac tissue.

52. The method of any one of the preceding claims, wherein said hPSCs are embryonic stem cells.

53. Engineered human cardiac tissue comprising at least about 30% hPSC-derived cardiomyocytes, about 35% or less hPSC-derived stromal cells/fibroblasts and up about 30% or less hPSC-derived endothelial cells, wherein the aforementioned cells are located throughout a hydrogel composition.

54. Engineered human cardiac tissue comprising hPSC-derived cardiomyocytes, hPSC-derived stromal cells/fibroblasts and hPSC-derived endothelial cells, wherein the aforementioned cells are derived from a single differentiation process, wherein the aforementioned cells are located throughout a hydrogel composition.

55. The engineered human cardiac tissue of claim 53 or 54, wherein the engineered human cardiac tissue further comprises vascular smooth muscle cells and cardiac progenitor cells.

56. The engineered human cardiac tissue of any one of claims 53 to 55, wherein the engineered human cardiac tissue comprises:
about 30 to about 70% cardiomyocytes;
about 10 to about 35% stromal cells/fibroblasts; and
about 5 to about 30% endothelial cells.

57. The engineered human cardiac tissue of any one of claims 53 to 56, wherein up to about 10% of the cardiomyocytes in said engineered human cardiac tissue are proliferative.

58. The engineered human cardiac tissue of any one of claims 53 to 57, wherein the engineered human cardiac tissue displays glycolytic metabolism.

59. The engineered human cardiac tissue of claim 53, wherein cell culture medium obtained following 24 h culture with said engineered human cardiac tissue has a concentration of lactate of 1 mM.

60. The engineered human cardiac tissue of any one of claims 53 to 59, wherein said engineered human cardiac tissue displays log2 relative gene expression of >2 of PKM when normalised to TUBA1A.

61. The engineered human cardiac tissue of any one of claims 53 to 60, wherein said engineered human cardiac tissue has tolerance to hypoxia.

62. The engineered human cardiac tissue of any one of claims 53 to 61, wherein said engineered human cardiac tissue yields a lactate/pyruvate ratio of at least 1:1 in cell culture medium obtained following 20 h of culture under hypoxic conditions.

63. The engineered human cardiac tissue of any one of claims 53 to 62, wherein said engineered human cardiac tissue displays log2 relative gene expression of <−4 of ALDH1, AGMO, GPX3, CYP1B1 and PLIN5 when normalised to TUBA1A expression.

64. The engineered human cardiac tissue of any one of claims 53 to 63, wherein the cardiomyocytes are MMP1⁺.

65. The engineered human cardiac tissue of any one of claims 53 to 64, wherein the fibroblasts are MMP1⁺, EMP1⁺, FOXD1⁺, RAB27B⁺, NR2F1⁺, F2RL1⁺, SPP1⁺, TMEM158⁺, PTHLH+, PHLDA2⁺, MALL⁺, MYCT1⁺, DUSP4⁺, PLAU⁺, TMEM156⁺, CD274⁺, MMP10⁺, ARRDC4⁺, RFX8⁺, MLPH⁺, THBD⁺, HHEX⁺, VGF⁺, OTULINL⁺, IL33⁺, CA12⁺, C6orf141⁺, MFSD2A⁺ and/or CARD10⁺.

66. The engineered human cardiac tissue of any one of claims 53 to 65 comprising:
about 30 to about 70% cardiomyocytes;
about 10 to about 35% stromal cells/fibroblasts;
about 5 to about 30% endothelial cells;
up to about 15% vascular smooth muscle cells; and
up to about 10 % cardiac progenitor cells.

67. The engineered human cardiac tissue of any one of claims 53 to 66, wherein said engineered human cardiac tissue exhibits contractile activity of less than 10 mN/mm².

68. The engineered human cardiac tissue of any one of claims 53 to 67, wherein the hydrogel composition comprises fibrin formed by mixing thrombin and fibrinogen solutions.

69. The engineered human cardiac tissue of claim 68, wherein fibrinogen is present at a concentration of 10 mg/mL-50 mg/mL.

70. The engineered human cardiac tissue of claim 69, wherein fibrinogen is present at a concentration of 20 mg/mL.

71. The engineered human cardiac tissue of any one of claim 53 to 70, wherein the total number of cells in the tissue is about 20×10⁶ cells to 100×10⁶ cells.

72. The engineered human cardiac tissue of any one of claims 53 to 71, wherein the engineered human cardiac tissue is in the form of a tissue patch.

73. The engineered human cardiac tissue of any one of claims 53 to 72, wherein said hPSCs are iPSCs.

74. The engineered human cardiac tissue of any one of claims 53 to 73, wherein said hPSCs are embryonic stem cells.

75. Engineered human cardiac tissue produced according to the method of any one of claims 1 to 52.

76. A method for analysing the biological effect of at least one test compound or bioactive agent on cardiac cells, comprising contacting engineered human cardiac tissue of one of claims 53 to 75 with the test compound or bioactive agent, incubating the tissue in the presence of the test compound or bioactive agent, and analysing the biological effect.

77. Engineered human cardiac tissue of one of claims 53 to 75 for use as an implant in the treatment of diseased or damaged cardiac tissue in a subject in need thereof.

78. A method for treating diseased or damaged cardiac tissue in a subject in need thereof comprising implanting the engineered human cardiac tissue of one of claims 53 to 75 in said subject.

79. Use of engineered human cardiac tissue of one of claims 53 to 75 in the manufacture of a medicament for the treatment of diseased or damaged cardiac tissue in a subject in need thereof.

80. The engineered human cardiac tissue of claim 77, the method of claim 78 or the use of claim 79, wherein the subject is suffering from cardiomyopathy or a cardiac tissue injury.

81. The engineered human cardiac tissue, the method or the use of claim 80, wherein the cardiomyopathy or cardiac tissue injury is due to acute or chronic stress, atheromatous disorders of blood vessels, ischemia, myocardial infarction, inflammatory disease, heart valve disease, or myocarditis.

82. The engineered human cardiac tissue of claim 77, the method of claim 78 or the use of claim 79, wherein the subject is suffering from a congenital heart disease.

83. The engineered human cardiac tissue, the method or the use of claim 82, wherein the congenital heart disease is selected from a group consisting of single ventricle disorders including hypoplastic left heart syndrome, tetralogy of fallot, truncus arteriosus, pulmonary atresia, ventricular septal defects, atrial septal defects, and endocardial cushion defect.
